(19) United States
(12) Patent Application Publication
(10) Pub. No.: US 2020/0239861 A1
(43) Pub. Date: Jul. 30, 2020

(54) MICROSOMES IRREVERSIBLY INHIBITED FOR CYP450 THEIR USES IN THE PHENOTYPING OF ENZYMATIC METABOLIC PATHWAYS

(71) Applicant: LES LABORATOIRES SERVIER, Suresnes Cedex (FR)

(72) Inventors: Fabrice CARADEC, Donnery (FR); Yannick Parmentier, Saint-Denis-En-Val (FR); Corinne Pothier, Saint-Jean-De-Braye (FR)

(21) Appl. No.: 16/851,551

(22) Filed: Apr. 17, 2020

Related U.S. Application Data
(63) Continuation of application No. 15/264,786, filed on Sep. 14, 2016, now abandoned.
Application No. 15/264,786 is a continuation of application No. 14/561,817, filed on Dec. 5, 2014, now abandoned.
(60) Provisional application No. 61/912,186, filed on Dec. 5, 2013.

(30) Foreign Application Priority Data
Dec. 5, 2013 (EP) 13306674.6

(51) Int. Cl. C12N 9/02; C12Q 1/26

(52) U.S. Cl. CPC C12N 9/0071; C12N 9/0073; G01N 2333/90245; C12Y 114/00; G01N 2333/90209; C12Q 1/26

(57) ABSTRACT

A method of preparing isolated microsomes comprising an irreversibly inhibited cytochrome P450 (CYP450). Isolated microsomes are characterized in that a cytochrome P450 thereof is irreversibly inhibited by a non-reversible inhibitor. The isolated microsomes according to the invention may be used in a method of phenotyping enzymatic reactions of a drug candidate.

The Invention Is located in the field of research and development of products and methods for the evaluation of drug interactions in the case of new drugs. The present invention relates to a method of preparing isolated microsomes irreversibly inhibited for a specific human cytochrome P450 (CYP450), which microsomes will be used in quantifying the contribution of that enzyme to the metabolism of active ingredients.

The efficacy and toxicity of a drug can be modified by administration of another compound: drug, environmental pollutant, foodstuff. These are drug Interactions (DDI; Drug-Drug Interactions). Different types of Interaction mechanisms exist, the most important being the metabolic Interaction which belongs to the group of pharmacokinetic interactions.

A metabolic drug Interaction is understood to be, inter alia, the fact that a drug A can modify the metabolism of a co-administered drug B either by accelerating the metabolism of B (activation or Induction) or by reducing it (Inhibition or repression). This metabolic drug interaction will, in the development program for a new drug, necessitate identification of the enzymes involved in the metabolism of the active ingredient, on the one hand, and of the potential inhibitor or inducer of said active ingredient, on the other hand.

The liver is the main site for the metabolism of drugs. The hepatocyte contains the essential enzymes for metabolism, including cytochromes P450 (CYP450). Cytochromes P450 accordingly constitute the main target in the prediction of drug interactions.

In order to predict the risk of drug Interactions it is necessary to identify and determine the contribution of each enzyme to the metabolism of the active Ingredient; this is the phenotyping of enzymatic reactions.

In order to estimate the contribution of each enzyme to the metabolism of the active Ingredient, various methods of phenotyping the metabolic pathways can be employed.

The use of a bank of characterised human liver microsomes can assist in identifying enzymes involved in the metabolism of an active ingredient. This method requires prior characterisation of the enzymatic activity of the main CYP450s from at least fifteen individual batches of microsomes obtained from human livers. Each of the fifteen batches of microsomes is Incubated with the active ingredient in order to determine a correlation between the rate of its metabolism and the activity of each cytochrome P450 of those same microsomes. However, the use of microsome banks does not allow quantitative determination of the enzymes Involved so that the correlation is difficult to perform.

Recombinant enzymes are also used in order to estimate the relative contribution of each cytochrome P450 to the metabolism of the active Ingredient. As the level of expression of the cytochrome P450 in the recombinant system is different, often being very much Increased compared to native human liver microsomes, it is necessary to incorporate a correction factor in order to extrapolate the contribution of each CYP450 in recombinant microsomes compared to human microsomes (correction factor a Relative Activity Factor). This correction factor has to be calculated by experimental measurement of the enzymatic activity of each CYP450 tested, firstly, in the presence of native human microsomes and, secondly, in the presence of recombinant microeomes. This approach makes it possible to indirectly evaluate, in selective and semi-quantitative manner, the relative contribution of each enzyme to the metabolism of the active ingredient. However, owing to their intrinsic characteristics, these recombinant enzymes differ from the enzymes found in liver microsomes (truncated protein sequence, different membrane environment, different coupling between cytochromes b5 and P450, absence of competition between the various CYP450s).

Biological inhibitors, such as monoclonal antibodies, directed against an enzyme are used after co-Incubation of the active Ingredient with human liver microsomes (versus a control without antibodies) for a quantitative estimate of the metabolism of the active Ingredient. However, a significant number of the antibodies used show a lack of specificity and inhibitory power. Finally, this technique is cumbersome to employ for the phenotyping of metabolic pathways.

Chemical inhibitors of CYP450s make it possible to determine the contribution of each cytochrome P450 directly by means of the percentage inhibition of the metabolism of the active ingredient if the inhibition is total and specific for the enzyme studied. The inhibitors may bind reversibly or non-reversibly to the enzyme. They are used in co-incubation or pre-incubation with the active Ingredient and human liver microsomes (compared to a control condition without inhibitor)—a representative model of the in vivo situation for the oxidative metabolism pathways of active Ingredients.

The level of Inhibition associated with competitive-type reversible inhibitors (the most frequent type) is dependent on incubation conditions such as Incubation time and substrate concentration. In addition, many inhibitors of CYP450 commonly used for phenotyping studies are not specific for a single CYP450 (e.g.: ketoconazole, quercetine . . . ). Consequently, reversible inhibitors are not suitable for phenotyping cytochromes P450.

In order to remedy the drawbacks of those methods, non-reversible or irreversible inhibitors are used in order to obtain a robust quantitative method which is representative of in vivo conditions. The inhibition is said to be irreversible as the enzyme never recovers its activity; reference is made to “suicide” inhibition. Non-reversible inhibitors are oxidised by cytochromes P450 to form intermediate metabolites which bind Irreversibly to the enzymes. This process is termed MBI, standing for Mechanism-Based Inhibition, because the starting compound is not inhibitory but requires at least one catalytic cycle of the enzyme before being activated into a to reactive metabolite which binds covalently to said enzyme. MBI inhibition is characterised by Irreversible inhibition of the cytochrome P450 studied and does not depend on the concentration of substrate. These inhibitors follow first-order kinetics with the following constants: k_{inact} corresponds to the maximum enzyme inactivation rate and Kᵢ corresponds to the concentration is of Inhibitor at half the maximum inactivation rate.

Several conditions have to be combined in order to obtain total inhibition of a CYP450 using an Irreversible inhibitor:

1) It Is necessary to use a sufficient concentration of non-reversible inhibitor (depending on its Kᵢ);

2) depending on its k_{inact}, the time period of pre-incubation of the Inhibitor with the liver microsomes must be sufficient to bring about sufficient catalytic Inactivation cycles;

3) the time period of pre-incubation must not bring about depletion of the suicide Inhibitor such that Its concentration would become too low to totally inhibit the P450.

If inhibition of the CYP450 is total and specific (no other P450 is inhibited) It is therefore possible to quantitatively determine the involvement of each cytochrome P450 in metabolism of an active ingredient. The experimental arrangement used is then as follows:

| Sequence | | Liver microsomes | 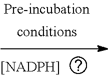 | Irreversibly inhibited microsomes | 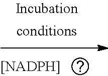 | f(t) = [AI] |
|---|---|---|---|---|---|---|
| Active | Test | + MBI | | + AI | | |
| Ingredient test | Control | − MBI | | + AI | | |
| Control + | Test | + MBI | | + substrate | | |
| specific | Control | − MBI | | + substrate | | |
| substrate | | | | | | |

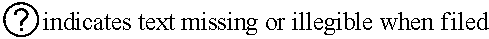

Despite the above-mentioned advantages, the use of MBIs according to this experimental arrangement has a certain number of constraints which limit its interest:
On the one hand, the Incubation conditions of the active ingredient studied are dependent on optimum conditions for pre-Incubation of the non-reversible Inhibitor (concentrations of microsomal proteins, percentage of organic solvent). Indeed, in order to precisely measure the percentage inhibition of the to metabolism of the active ingredient, the latter must be incubated under so-called initial conditions (linearity of the rate of the metabolism as a function of time and protein concentrations; the percentage solvent must not exceed a certain level). In order to obtain maximum and specific Inhibition in this linear experimental is arrangement, the inhibitor must itself be previously incubated under in vitro conditions of concentrations of proteins or solvent which are compatible with Incubation of the active ingredient.
On the other hand, the pre-incubation time alters the enzymatic activity of the CYP450s of microsomes, which have an in vitro half-life of about 70 to 90 minutes. For example, a loss of CYP2D6 enzymatic activity of up to 26% Is observed in the case of pre-incubation for 30 minutes, up to 35% for pre-incubation of 40 minutes and 46% for pre-incubation of 60 minutes. Consequently, the duration of the continuous sequence of pre-incubation of the MBI and incubation of the drug candidate imposed by the experimental arrangement is therefore incompatible with the in vitro half-life of the CYP450s of microsomes.
Finally, it is often the case that Irreversible Inhibitors for one CYP450 also exhibit reversible inhibition for one or more CYP450 (for example, the MBI inhibitor of CYP1A2 is also a competitive inhibitor of CYP2C19 and the MBI Inhibitor of CYP2B6 is also a competitive inhibitor of CYP2A6 and 3A4). So the above linear experimental arrangement is not capable of satisfactorily eliminating, for an in vitro phenotyping study, the remaining free inhibitor portion which is capable of acting competitively on other CYP450s. Dilution of the pre-incubate can reduce the concentration of free inhibitor, resulting in less non-specific is reversible inhibition, but this will, de facto, reduce the concentration of microsomal proteins in such a way that the metabolism of the active ingredient during the incubation will be too weak to be detected.

Consequently, the difficulties in making the incubation conditions for, on the one hand, the irreversible inhibitor compatible with those for, on the other hand, the active ingredient are such that it is seldom possible to use that linear experimental arrangement.

In addition, in the case of that linear experimental arrangement, it is necessary to add two series of additional incubations (with and without Irreversible inhibitor) with a specific substrate of the CYP450 tested as a positive control validating the effect of the inhibitor. This therefore requires that not only the active ingredient be quantified but also the entirety of the specific substrates, this being the case for as many CYP450s as there are to be tested, which makes the experiment considerably more onerous.

In view of the respective drawbacks of the methods of phenotyping the enzymatic reactions involved in the metabolism of an active ingredient, an in vitro study of said phenotyping of enzymatic reactions requires that the disadvantages of the various previously described methods be overcome.

The aim of the present invention is therefore to propose an alternative strategy which makes it possible to overcome the problems inherent in implementing a phenotyping study of the enzymatic reactions involved in the metabolism of an active Ingredient by the use of isolated Irreversibly inhibited microsomes.

The Invention relates to a method of preparing isolated microsomes having an Irreversibly inhibited cytochrome P450 (CYP450) and comprising the following steps:
irreversible inhibition of the cytochrome P450;
concentration of the microsomal proteins.

A non-reversible inhibitor, irreversible Inhibitor, MBI inhibitor, irreversible inhibition or suicide inhibition are understood to refer to an inhibitor which Is capable of covalently binding to an enzyme; the enzyme so inhibited cannot regain its initial functional activity. More specifically, an MBI (Mechanism Based Inhibition) inhibitor does not form an irreversible bond with the enzyme directly but rather one of its reactive intermediate metabolites bonds covalently to the enzyme.

The aim of concentration in accordance with the invention is, on the one hand, to filter off the microsomal proteins from the other products present in the preparation, especially the excess Irreversible inhibitor that remains free and the solvents, and, on the other hand, to concentrate the microsomes diluted by the pre-incubation step.

Concentration of the microsomal proteins in accordance with the invention can be obtained by filtration and then centrifugation. The filtration function in the concentration step is obtained starting from a membrane having a cut-off threshold of from 10,000 daltons to 40,000 daltons, preferably 30,000 daltons. The concentration function in the concentration step is carried out by a system which enables concentration, for example a Centricon® system subjected to centrifugation of from 3000 g to 4000 g for from 60 to 90 minutes, preferably for 80 minutes. At the end of that step, the Centricon® is reversed and centrifuged at from 800 to 1000 g for from 2 to 10 minutes, preferably for 5 minutes.

In order to Improve separation and concentration of the microsomal proteins, an additional step of ultracentrifugation under the following conditions of from 80,000 g to 150,000 g for about 60 minutes can be carried out.

Concentration of the microsomal proteins can also be Improved by repeating at least twice the above-described sequence: filtration and then concentration.

The step of concentration of the microsomal proteins according to the invention can also be obtained by successive ultracentrifugations. At least two successive ultracentrifugations are necessary under the following conditions of from 80,000 g to 150,000 g for from 60 to 90 minutes.

The invention relates to a method of preparing isolated irreversibly inhibited microsomes in which one or more washing steps are added. This method of preparing isolated microsomes includes washing steps placed before and/or after the step of concentration of the microsomal proteins. For example, the method according to the Invention comprises the following steps;
irreversible Inhibition of a cytochrome P450;
washing;
concentration of the microsomal proteins;
washing of the microsomal proteins.

The method according to the invention may also comprise the following steps:
irreversible inhibition of a cytochrome P450;
concentration of the microsomal proteins;
washing of the microsomal proteins;

or the following steps:
irreversible inhibition of a cytochrome P450;
washing;
concentration of the microsomal proteins.

Washing is understood to mean a step of rinsing and removal of non-microsomal fractions. The washing step may also consist of taking up the microsome concentrate in Tris HCl buffer pH 7.4.

At the end of the method of preparing isolated irreversibly inhibited microsomes in accordance with the Invention, the microsomes are at a concentration of from 10 mg/ml to 30 mg/ml, more specifically from 17 mg/ml to 25 mg/ml, and preferably 20 mg/ml, the microsomal proteins having been concentrated by a factor of from 5 to 15.

The method of preparing isolated Inactivated microsomes according to the present invention comprises a final step of preservation of the microsomes. The step of preservation of the microsomes consists preferably of a step of cryopreservation consisting of cooling the isolated and purified microsomes to a very low temperature (about −196° C.), in the presence—or not—of cryoprotective solutions, in order to suspend any biological activity. The aim of this preservation step is to facilitate the extemporaneous use of the inactivated and isolated microsomes according to the invention. This use of the isolated microsomes may take place from a few days to several months after their preparation. The preservation step in no way alters the structural and functional properties of the microsomes.

Preferably, the preservation step is a freezing step.

Freezing of the irreversibly inhibited and isolated microsomes is carried out at −80° C.

The microsomes used in the course of the method of preparation are human, rat, mouse, pig or monkey liver microsomes. The microsomes are to preferably obtained from human livers containing all the P450 enzymes. In order to take account of Inter-individual variability, the microsomes come from several donors in order to form a pool of microsomes.

The cytochromes P460 are a huge family of Isoenzymes composed of 18 is sub-families (CYP1, CYP2, CYP3, CYP4, CYP5, CYP7, CYP8, CYP11, CYP17, CYP19, CYP20, CYP21, CYP24, CYP26, CYP27, CYP39, CYP46, CYP51). The present invention relates to a method of preparing isolated microsomes where the Irreversibly inhibited cytochrome P450 is selected from the cytochrome families CYP1, CYP2, CYP3 and CYP4.

The irreversibly inhibited cytochrome P450 is selected from CYP1A1, CYP1A2, CYP2A6, CYP286, CYP2C8, CYP2C9, CYP2C19, CYP2D6, CYP2E1, CYP2J2, CYP3A4, CYP3A5 and CYP4F2.

The irreversibly inhibited cytochrome P450 is preferably selected from CYP1 A2, CYP2A6, CYP2B6, CYP2C8, CYP2C9, CYP2C19, CYP2D6. CYP2E1 and CYP3A4.

Among the non-reversible inhibitors used in the method of preparing microsomes according to the invention there may be mentioned, by way of Illustration and without Implying any limitation:

| | | In vitro parameters (literature) | |
|---|---|---|---|
| CYP | MBI inhibitors | K₁ (μM) | k_{inact} (min−1) |
| 1A2 | Furafylline | 3-6 | 0.27-0.87 |
| 2A6 | 8-MethOxyPsoralen | 0.3-1.9 | 0.5-2.1 |
| | Menthofuran | 0.84-2.5 | 0.22-0.25 |
| | Psoralen | 0.6 | 0.3 |
| 2B6 | Thiotepa | 4.8-50 | 0.1-0.2 |
| | Phencyclidine | 10 | 0.01 |
| 2C8 | Gemfibrozil gluc. | 50 | 0.21 |
| 2C9 | Tienilic acid | 0.13-20 | 0.05-2 |
| | Suprofen | 3.7-26 | 0.07-0.09 |
| 2C19 | Fluoxetine | 0.2-54 | 0.06-0.1 |
| | Clopidogrel | 0.5-14.3 | 0.056-0.35 |
| | Ticlopidine | 1.65-87 | 0.19-0.032 |
| | Protopine | 7.1 | 0.24 |
| 2D6 | Paroxetine | 0.8-9.32 | 0.17 |
| | MDMA | 23 | 0.18 |
| | EMTPP | 5.5 | 0.09 |
| 2E1 | DiethylDithioCarbamate | 12.2 | 0.02 |
| | Phenethyl isothiocyanate | 9.98 | 0.33 |
| 3A4 | TroleAndOmycine | 2.4 | 0.032 |
| | Diltiazem | 8.7 | 0.005 |
| | Azamulin | 0.2 | 0.7 |

The present invention relates also to Isolated microsomes per se, comprising an Irreversibly inhibited cytochrome P450.

The isolated microsomes according to the invention comprise an irreversibly inhibited cytochrome selected from the cytochrome families CYP1, CYP2, CYP3 and CYP4.

The invention relates to isolated microsomes wherein a cytochrome selected from the following list Is irreversibly inhibited: CYP1A1, CYP1A2, CYP2A6. CYP2B6, CYP2C8, CYP2C9, CYP2C19, CYP2D6, CYP2E1, CYP2J2, CYP3A4, CYP3A5 and CYP4F2.

More especially, the Invention relates to isolated microsomes wherein a cytochrome selected from the following list is irreversibly inhibited: CYP1A2, CYP2A6, CYP2B6, CYP2C8. CYP2C9, CYP2C19, CYP2D6, CYP2E1 and CYP3A4.

The isolated irreversibly inhibited microsomes according to the invention are preferably cryopreserved or cryoprotected.

The present invention relates also to isolated Irreversibly inhibited microsomes in frozen form for extemporaneous use after thawing.

The isolated microsomes are obtained in accordance with the preparation methods described in the present patent application.

The invention relates to use of the isolated irreversibly inhibited microsomes in the phenotyping of enzymatic reactions Involved in the metabolism of an active Ingredient to be evaluated.

The invention relates also to a method of phenotyping enzymatic reactions involved in the metabolism of an active ingredient. This phenotyping method comprises the following steps:
Incubation of microsomes isolated and irreversibly Inhibited in accordance with the invention with an active ingredient to be evaluated;
measurement of the contribution of the Irreversibly inhibited cytochrome P450 involved in the metabolism of the active ingredient.

Incubation is carried out in the presence of the active ingredient with, on the one hand, isolated irreversibly inhibited liver microsomes according to the invention and, on the other hand, non-inhibited isolated liver microsomes prepared according to the invention (control). Incubation Is monitored either at a final Incubation timepoint or in kinetic manner, which is to say at several Incubation timepoints.

At each incubation timepoint, the active ingredient and/or Its metabolites are quantified.

The metabolic activity (A) of the active ingredient is measured either via the intrinsic metabolic clearance (In the case of kinetic measurement) or via the speed of the disappearance of unchanged active ingredient or of the appearance of metabolites (if a final incubation timepoint has been selected) under the two conditions of inhibited (activity Ai) and non-inhibited (control with activity A).

The percentage inhibition of the metabolic activity of the active Ingredient is calculated as follows: percentage inhibition=(A−Ai)/A. The calculated percentage inhibition corresponds directly to the contribution of the irreversibly inhibited cytochrome P450 Involved in the metabolism of the is active ingredient.

Finally, the invention relates to a phenotyping kit comprising:
microsomes Isolated and Irreversibly Inhibited according to the invention;
control microsomes.

Control microsomes are understood to be microsomes having been subjected to the method of preparation according to the Invention in the absence of the irreversible Inhibitor.

Preferably, the phenotyping kit comprises, on the one hand, isolated microsomes that have been Irreversibly Inhibited and cryopreserved and, on the other hand, control microsomes, which also may be cryopreserved.

The present invention is illustrated by the following Figures and Examples without being limited thereby:

### BRIEF DESCRIPTION OF THE DRAWINGS

FIG. 1: Percentage inhibition of the activity of cytochromes P450 in irreversibly inhibited CYP microsomes.

The specific activities studied for each CYP450 correspond to the activities of phenacetin-O-deacetylase (CYP1A2, incubation of phenacetin at 4.5 μM), coumarin-7-hydroxylase (CYP2A6, incubation of coumarin at 2 μM), bupropion-hydroxylase (286, incubation of bupropion at 50 μM), paclitaxel-6α-hydroxylase (2C8, incubation of paclitaxel at 4 μM), diclofenac-4′-hydroxylase (2C9, incubation of diclofenac at 4 μM), omeprazole-5-hydroxylase (2C19, incubation of omeprazole at 5 μM), dextromethorphan-O-demethylase (206, incubation of dextromethorphan at 5 μM), chlorzoxazone-6-hydroxylase (2E1, incubation of chlorzoxazone at 40 μM) and testosterone-6p-hydroxylase, midazolam-1′-hydroxylase and nifedipine-reductase (3A4, incubation of testosterone at 30 μM, midazolam at 0.5 μM, nifedipine at 10 μM). The percentage inhibition is obtained by comparison of the P450 activities on microsomes irreversibly inhibited by furafylline and on control microsomes.

FIG. 2: Impact of furafylline at 5 μM and 10 μM on microsomes of human liver and on recombinant microsomes for CYP1A2.

FIG. 2A: Impact of furafylline at 5 μM and 10 μM on microsomes of human liver.

FIG. 2B: Impact of furafylline at 5 μM and 10 μM on recombinant microsomes for CYP1 A2.

FIG. 3: Percentage inhibition of the activity of cytochromes P450 in irreversibly inhibited CYP3A4 microsomes.

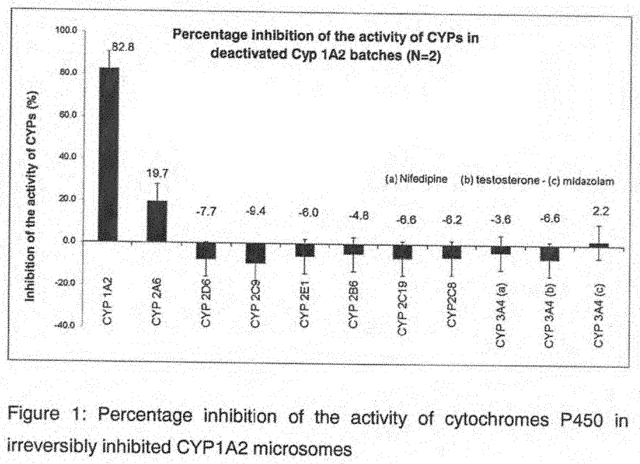

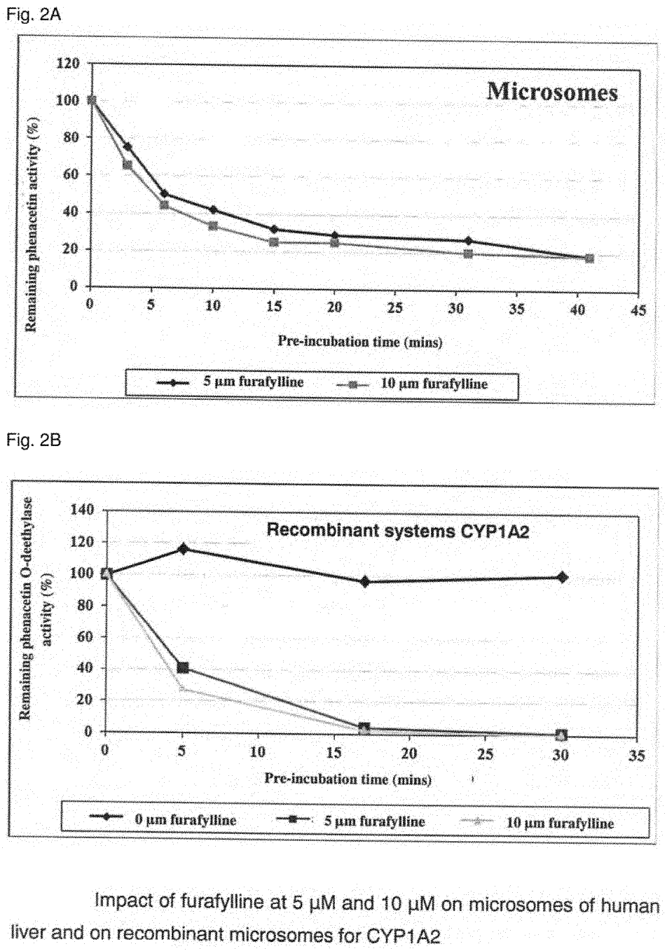

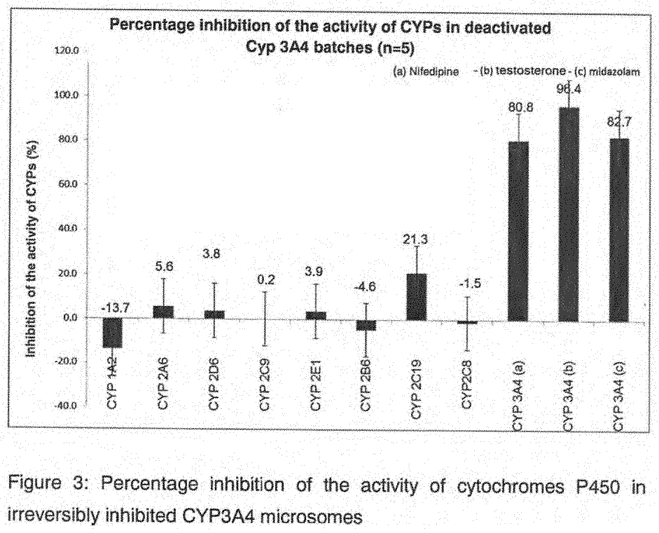

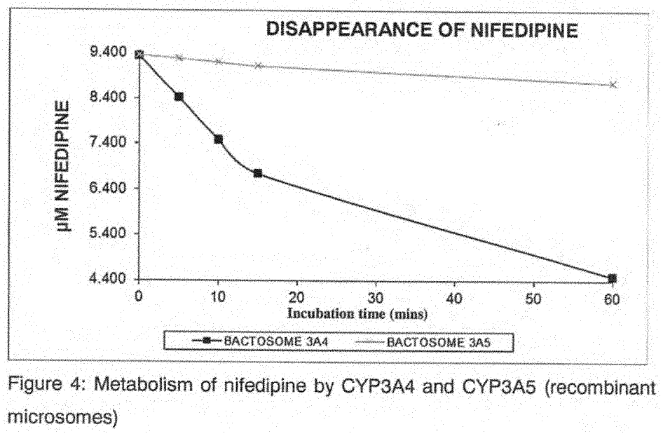

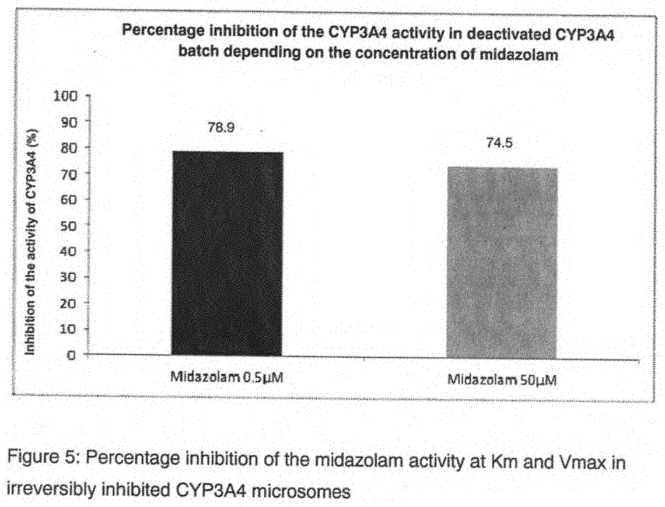

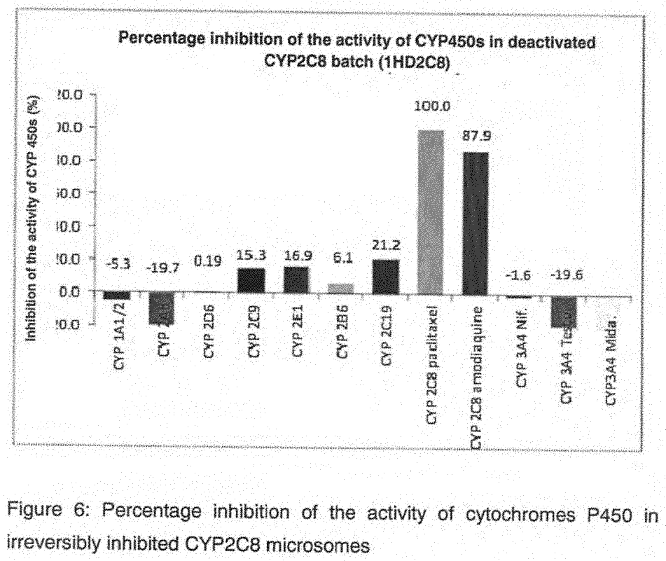

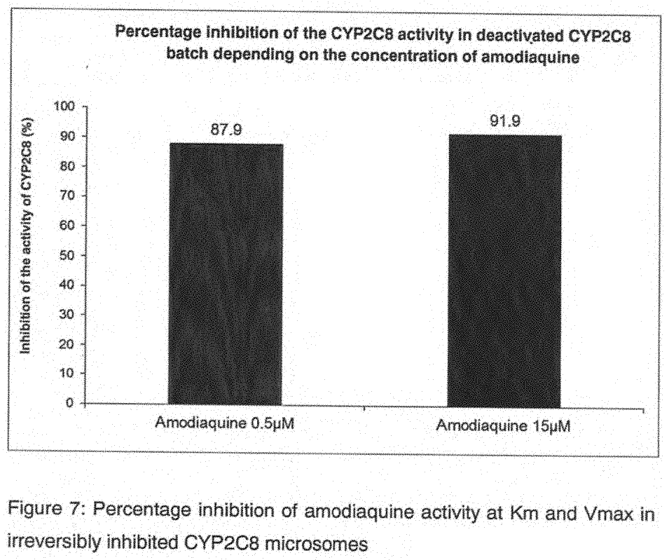

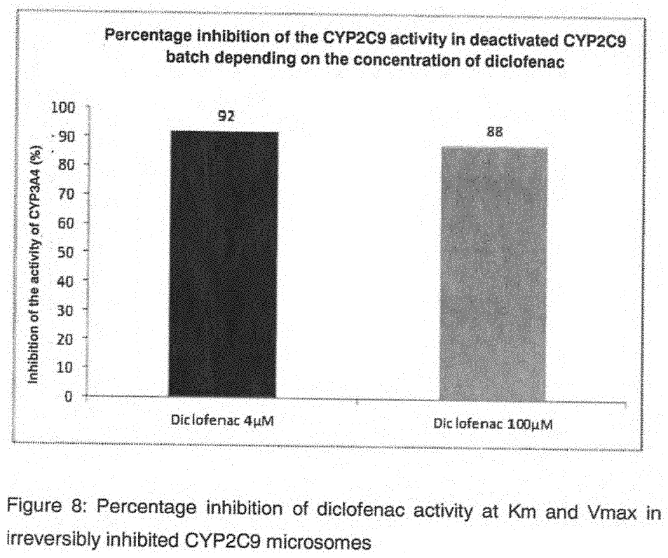

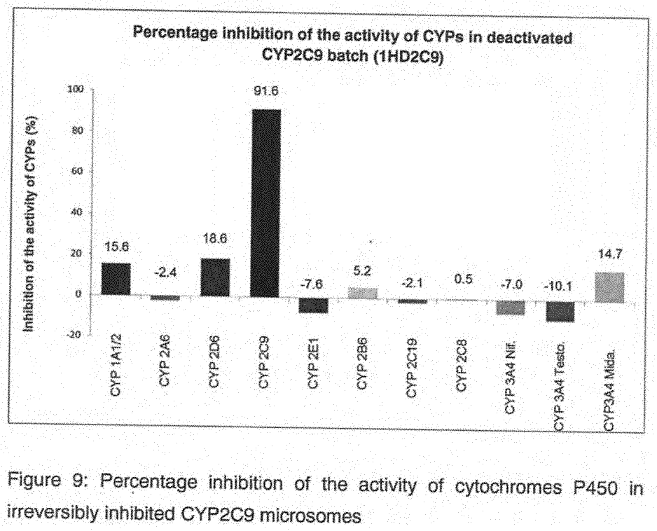

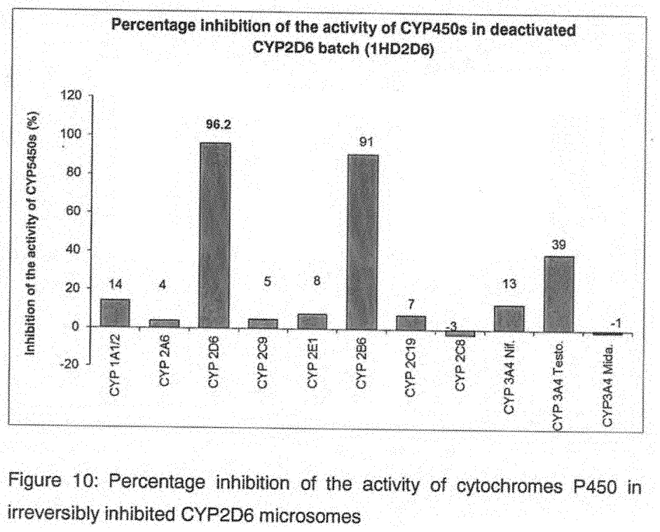

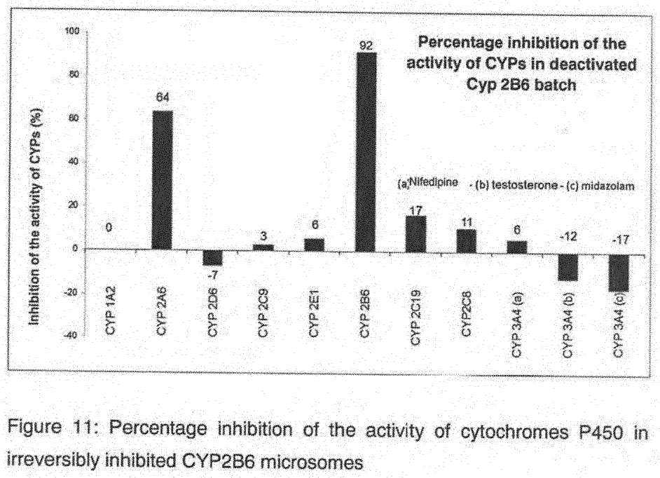

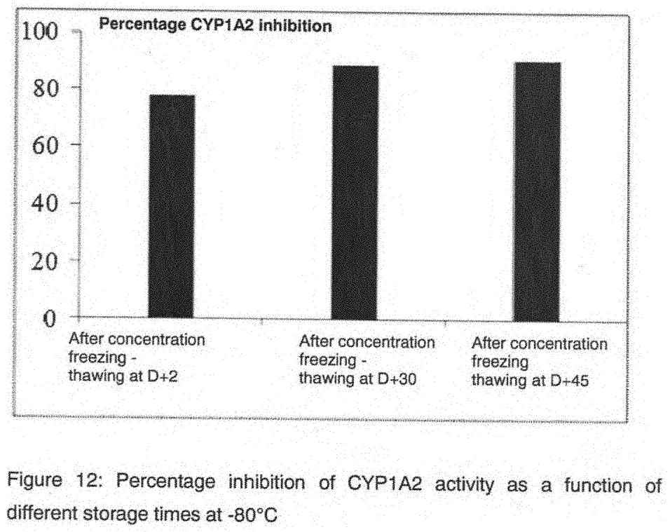

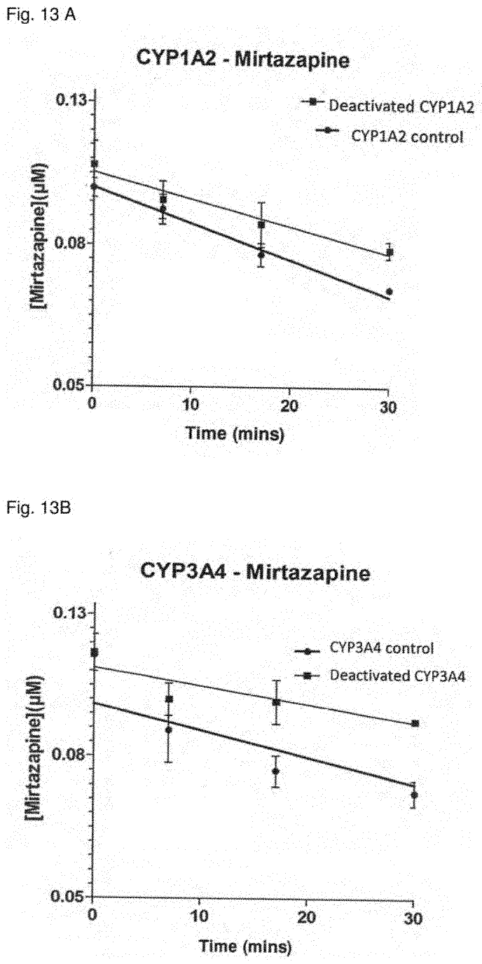

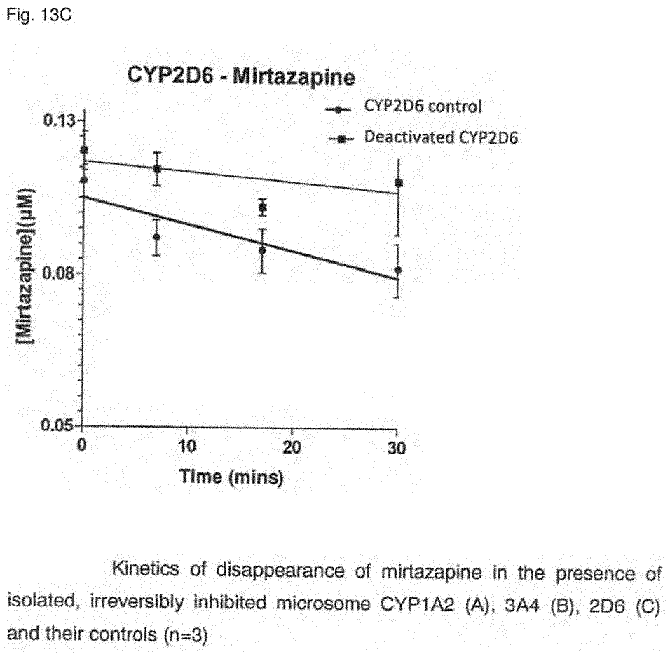

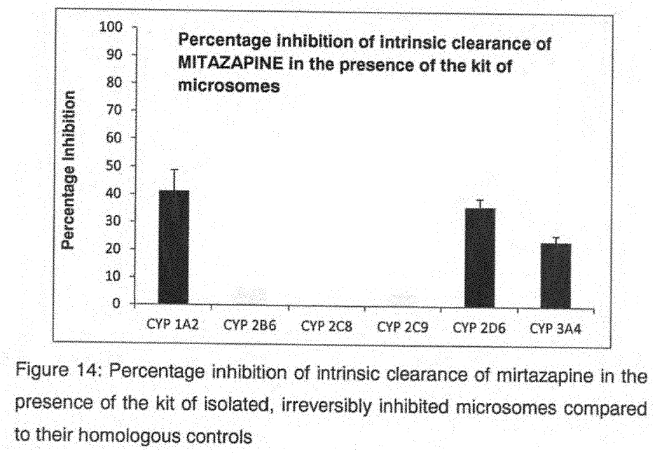

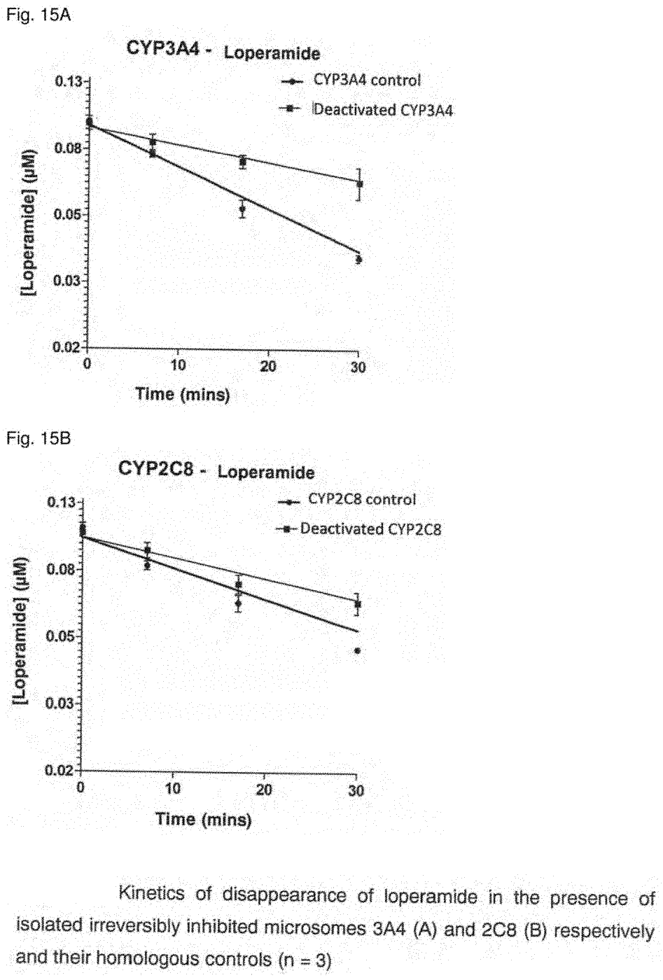

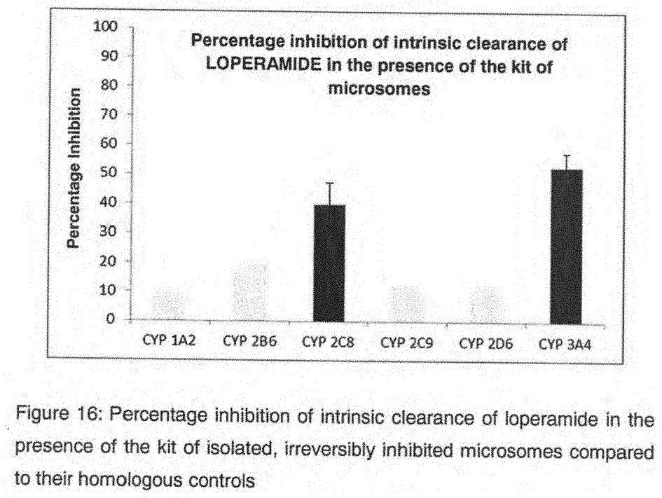

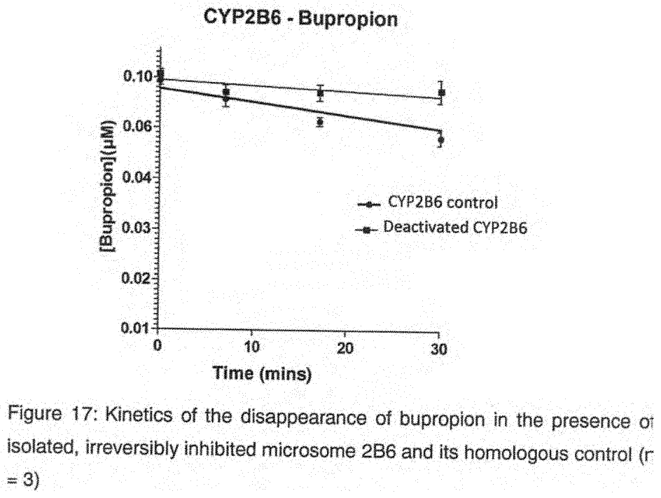

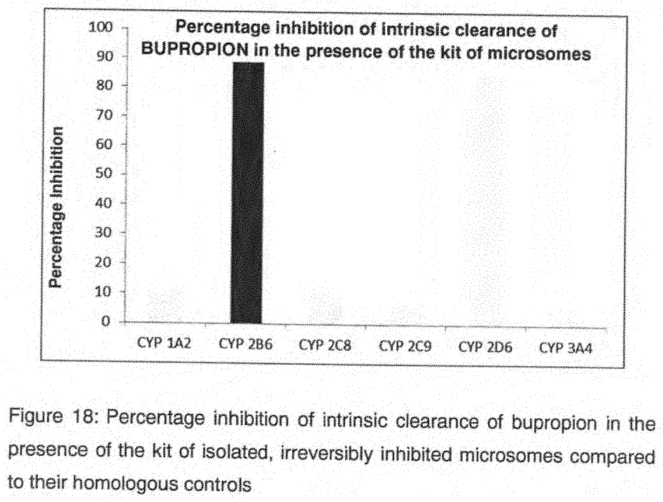

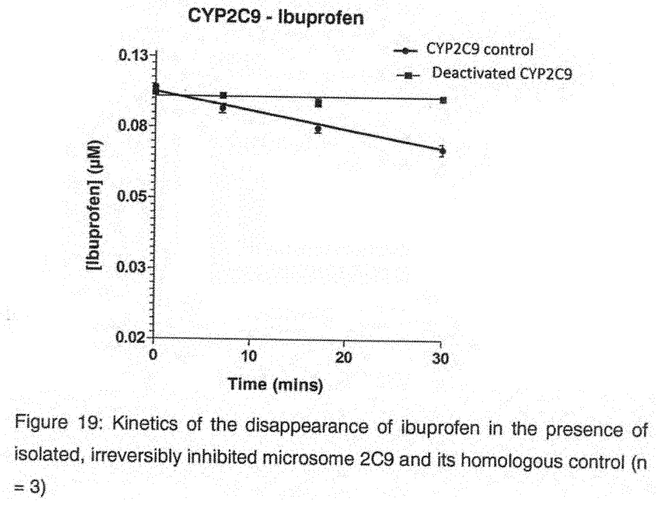

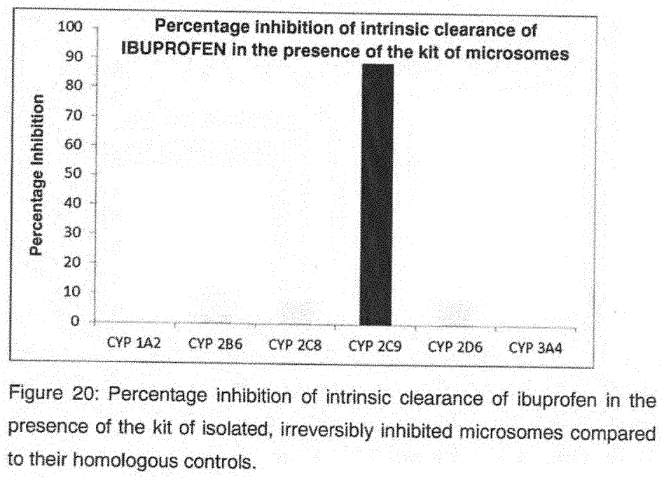

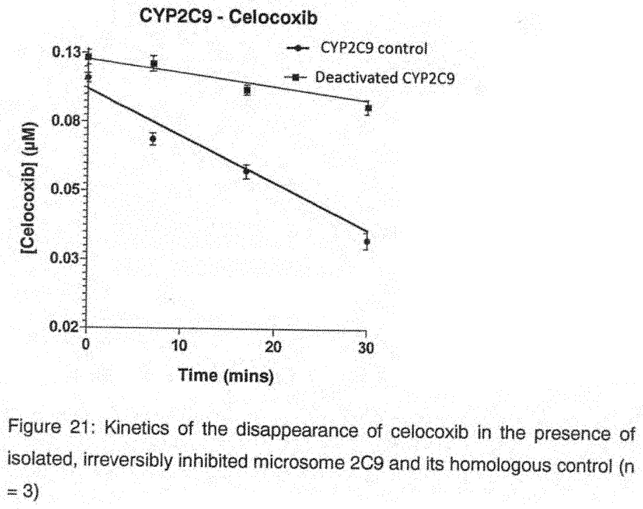

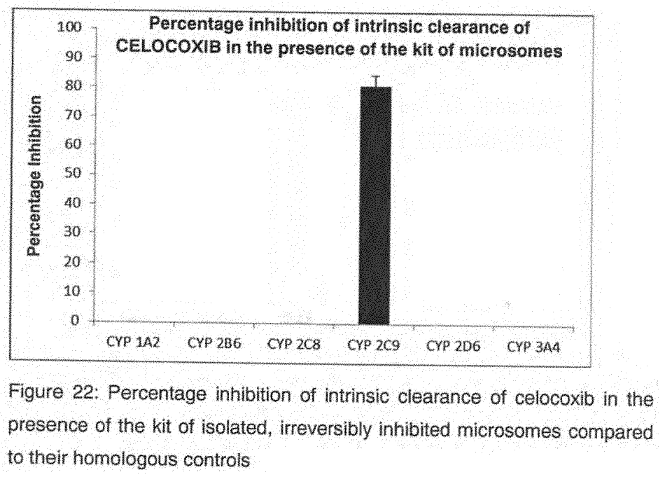

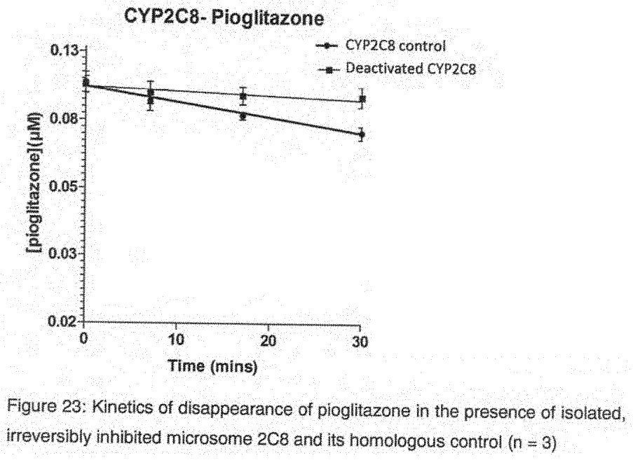

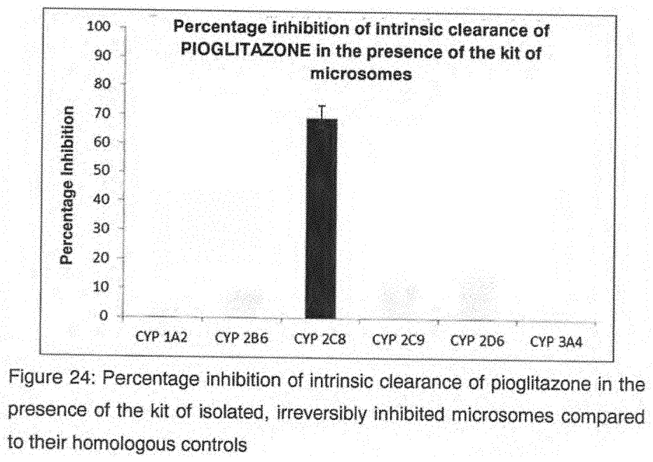

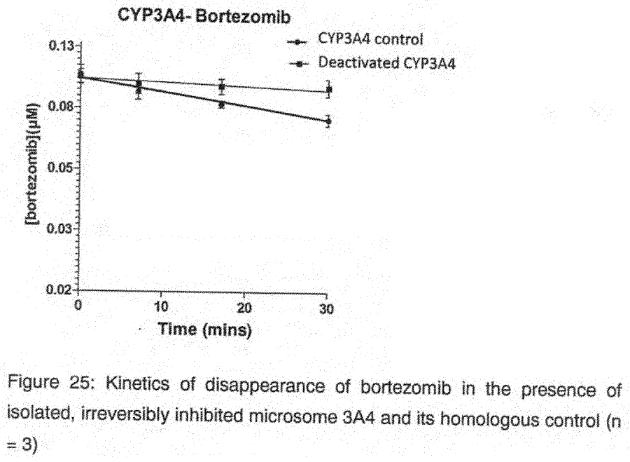

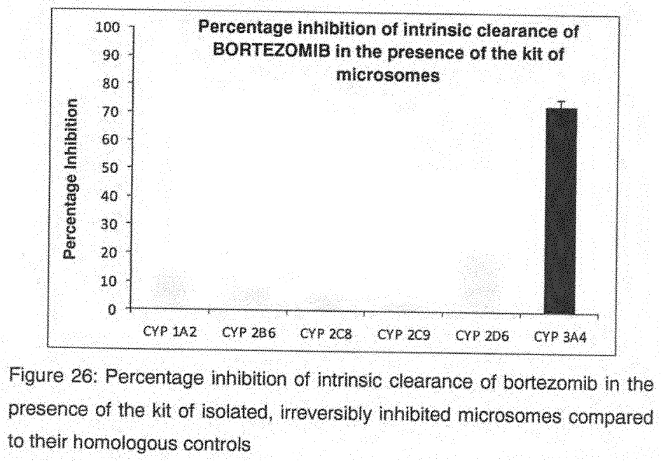

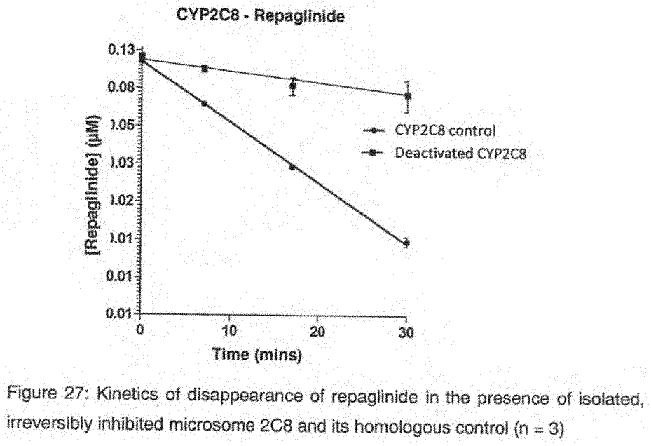

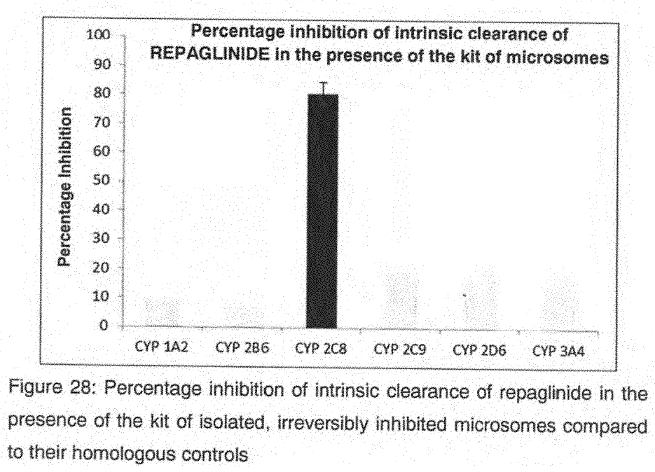

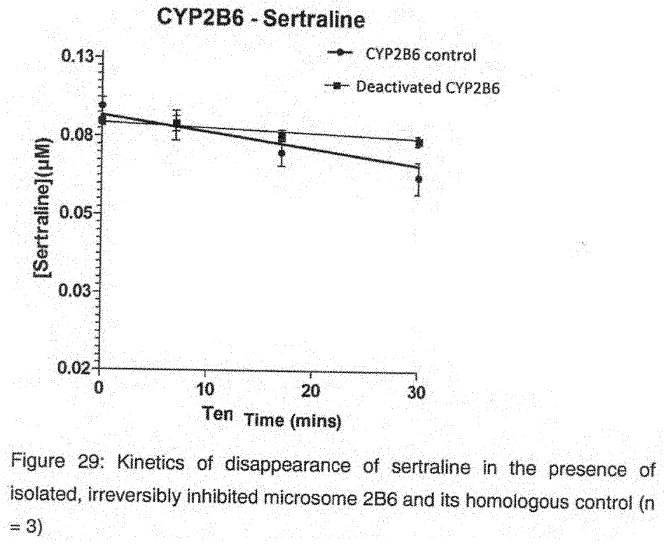

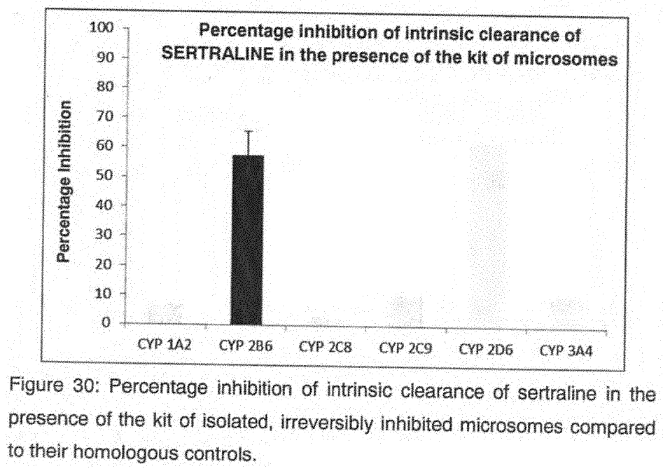

The specific activities studied for each CYP450 correspond to those described in FIG. 1. The percentage inhibition is obtained by comparison of the P450 activities on microsomes irreversibly inhibited by azamulin and on control microsomes.

FIG. 4: Metabolism of nifedipine and midazotam by CYP3A4 and CYP3A5 (recombinant microsomes).

FIG. 5: Percentage inhibition of the midazolam activity at Km and Vmax in irreversibly inhibited CYP3A4 microsomes.

FIG. 6: Percentage inhibition of the activity of cytochromes P450 in irreversibly inhibited CYP2C8 microsomes.

The specific activities studied for each CYP450 correspond to those described in FIG. 1 to which there is added amodiaquine hydroxylase (2C8, incubation of amodiaquine at 0.5 μM). The percentage inhibition is obtained by comparison of the P450 activities on microsomes irreversibly inhibited by gemfibrozil glucuronide and on control microsomes.

FIG. 7: Percentage inhibition of the activity of amodiaquine incubated at concentrations corresponding to the Km and Vmax of the 2C8-dependant reaction of amodiaquine hydroxylation, in irreversibly inhibited CYP2C8 microsomes.

FIG. 8: Percentage inhibition of the activity of diclofenac incubated at concentrations corresponding to the Km and Vmax of the 2C9-dependant reaction of diclofenac-4′-hydroxylation, in irreversibly inhibited CYP2C9 microsomes.

FIG. 9: Percentage inhibition of the activity of cytochromes P450 in irreversibly inhibited CYP2C9 microsomes.

The specific activities studied for each CYP450 correspond to those described in FIG. 1. The percentage inhibition is obtained by comparison of the P450 activities on microsomes irreversibly inhibited by tienilic acid and on control microsomes.

FIG. 10: Percentage inhibition of the activity of cytochromes P450 in irreversibly inhibited CYP206 microsomes. The specific activities studied for each CYP450 correspond to those described in FIG. 1. The percentage inhibition is obtained by comparison of the P450 activities on microsomes irreversibly inhibited by paroxetine and on control microsomes.

FIG. 11: Percentage inhibition of the activity of cytochromes P450 in irreversibly inhibited CYP2B6 microsomes.

The specific activities studied for each CYP450 correspond to the activities of phenacetin-O-deacetylase (CYP1A2, incubation of phenacetin at 200 μM), coumarin-7-hydroxylase (CYP2A6, incubation of coumarin at 20 μM), bupropion-hydroxylase (286, incubation of bupropion at 100 μM), amodiaquine-deethylase (2C8, incubation of amodiaquine at 20 μM), diclofenac-4′-hydroxylase (2C9, incubation of diclofenac at 200 μM), S-mephenytoin-hydroxylase (2C19, incubation of S-mephenytoin at 60 μM), dextromethorphan-O-demethylase (206, incubation of dextromethorphan at 100 μM), chlorzoxazone-6-hydroxylase (2E1, incubation of chlorzoxazone at 200 μM) and testosterone-60-hydroxylase, midazolam-1′-hydroxylase and nifedipine-reductase (3A4, incubation of testosterone at 75 μM, midazolam at 50 μM, nifedipine at 50 μM).

The percentage inhibition is obtained by comparison of the P450 activities on microsomes irreversibly inhibited by thioTEPA and on control microsomes.

FIG. 12: Percentage inhibition of CYP1A2 activity as a function of different storage times at −80° C.

FIG. 13: Kinetics of disappearance of mirtazapine in the presence of isolated, irreversibly inhibited microsome CYP1A2 (A), 3A4 (B), 206 (C) and their controls (n=3).

FIG. 13A: Kinetics of disappearance of mirtazapine in the presence of isolated, irreversibly inhibited microsome CYP1A2 and its control (n=3).

FIG. 13B: Kinetics of disappearance of mirtazapine in the presence of isolated, irreversibly inhibited microsome 3A4 and its control (n=3).

FIG. 13C: Kinetics of disappearance of mirtazapine in the presence of isolated, irreversibly inhibited microsome 206 and its control (n=3).

FIG. 14: Percentage inhibition of intrinsic clearance of mirtazapine in the presence of the kit of isolated, irreversibly inhibited microsomes compared to their homologous controls.

FIG. 15: Kinetics of disappearance of loperamide in the presence of isolated, irreversibly inhibited microsomes 3A4 (A) and 2C8 (B) respectively and their homologous controls (n=3).

FIG. 15A: Kinetics of disappearance of loperamide in the presence of isolated, irreversibly inhibited microsome 3A4 and its homologous control (n=3).

FIG. 15B: Kinetics of disappearance of loperamide in the presence of isolated, irreversibly inhibited microsome 2C8 and its homologous control (n=3).

FIG. 16: Percentage inhibition of intrinsic clearance of loperamide in the presence of the kit of isolated irreversibly inhibited microsomes compared to their homologous controls.

FIG. 17: Kinetics of the disappearance of bupropion in the presence of isolated, irreversibly inhibited microsome 286 and its homologous control (n=3).

FIG. 18: Percentage inhibition of intrinsic clearance of bupropion in the presence of the kit of isolated, irreversibly inhibited microsomes compared to their homologous controls.

FIG. 19: Kinetics of the disappearance of ibuprofen in the presence of isolated, irreversibly inhibited microsome 2C9 and its homologous control (n=3).

FIG. 20: Percentage inhibition of intrinsic clearance of ibuprofen in the presence of the kit of isolated, irreversibly inhibited microsomes compared to their homologous controls.

FIG. 21: Kinetics of the disappearance of celocoxib in the presence of isolated, irreversibly inhibited microsome 2C9 and its homologous control (n=3).

FIG. 22: Percentage inhibition of intrinsic clearance of celocoxib in the presence of the kit of isolated, irreversibly inhibited microsomes compared to their homologous controls.

FIG. 23: Kinetics of disappearance of pioglitazone in the presence of isolated, irreversibly inhibited microsome 2C8 and its homologous control (n=3).

FIG. 24: Percentage inhibition of intrinsic clearance of pioglitazone in the presence of the kit of isolated, irreversibly inhibited microsomes compared to their homologous controls.

FIG. 25: Kinetics of disappearance of bortezomib in the presence of isolated, irreversibly inhibited microsome 3A4 and its homologous control (n=3).

FIG. 26: Percentage inhibition of intrinsic clearance of bortezomib in the presence of the kit of isolated, irreversibly inhibited microsomes compared to their homologous controls.

FIG. 27: Kinetics of disappearance of repaglinide in the presence of isolated, irreversibly inhibited microsome 2C8 and its homologous control (n=3).

FIG. 28: Percentage inhibition of intrinsic clearance of repaglinide in the presence of the kit of isolated, irreversibly inhibited microsomes compared to their homologous controls.

FIG. 29: Kinetics of disappearance of sertraline in the presence of isolated, irreversibly inhibited microsome 286 and its homologous control (n=3).

FIG. 30: Percentage Inhibition of intrinsic clearance of sertraline in the presence of the kit of Isolated, irreversibly inhibited microsomes compared to their homologous controls.

### EXAMPLE 1: PREPARATION OF ISOLATED IRREVERSIBLY INACTIVATED MICROSOMES

Biological Materials

The microsomes are obtained from human livers containing all the P450 enzymes. They come from a pool of microsomes as they are obtained from several donors in order to take account of inter-individual variability.

Incubation of the Microsomes

For each preparation of batch of irreversibly inhibited microsomes, a control batch is prepared under the same conditions with the difference that the Irreversible inhibitor is replaced by an equivalent volume of solvent.

The non-reversible Inhibitor of the cytochrome P450 studied (or the solvent, for the control batch) is incubated with the microsomes in Tris/HCl buffer pH 7.4 and MgCl₂ with stirring at 37° C. The preparation is generally preheated for from 5 to 10 minutes and then NADPH is added to start the enzymatic reaction. At timepoint t, the reaction mixture is placed in ice for a few minutes before proceeding to the concentration step. Enzyme/Inhibitor complexes connected by covalent bonds are formed in the course of incubation of the microsomes with the Irreversible Inhibitor. The cytochrome P450 studied is inhibited in Irreversible, total and specific manner.

Filtration and Concentration of Irreversibly Deactivated Microsomes

The sample obtained from Incubation of the microsomes with the non-reversible inhibitor of the cytochrome P450 studied is filtered. This protein filtration step can be carried out using a membrane having a cut-off threshold of from 10,000 to 40,000 daltons. A Centricon® system is used to carry out this filtration step.

One or more washing steps is/are sometimes necessary to facilitate removal of the irreversible inhibitor that remains free. The sample Is to centrifuged at from 3000 g to 4000 g for 80 minutes and then from 800 to 1000 g for 5 minutes. The sample can undergo a succession of centrifugations in order to optimise the concentration of microsomal proteins.

Where appropriate, the concentrated sample of microsomes is then ultra-centrifuged in order to further improve the concentration of proteins. The ultracentrifugation is carried out under a range of conditions from 80,000 g over 4 hours to 150,000 g over 45 minutes, preferably at 100,000 g for 1 hour.

The protein concentrate is taken up in Tris/HCl buffer pH 7.4 and then aliquoted and frozen at −80° C.

At the end of this preparation there are obtained isolated Irreversibly inactivated microsomes that can be used extemporaneously for the phenotyping of enzymatic reactions involved in the metabolism of an active ingredient.

### EXAMPLE 2: CONDITIONS OF INHIBITION OF THE MAIN CYTOCHROMES P480 AND VALIDATION OF MICROSOMES IRREVERSIBLY INHIBITED ON SPECIFIC SUBSTRATES OF CYTOCHROMES P450

CYP1A2

Furafyline is one of the MBI inhibitors of the cytochrome CYP1A2.

The experimental conditions for maximum MBI inhibition by furafylline on CYP1 A2 are as follows:
microsomal proteins at 2 mg/ml;
furafylline at 10 μM;
pre-incubation time of 30 minutes.

After Incubation of phenacetin, a specific substrate of CYP1A2, at 4.5 μM (concentration less than or equal to its Km) with microsomes previously Inhibited under the conditions above, the percentage inhibition of the is phenacetin deacetylase activity (CYP1A1-/CYP1A2-dependant) is 83% (FIG. 1). It is to be noted that the remaining 17% metabolism Is due to the residual phenacetin deacetylase activity associated with CYP1A1 and not to a lack of inhibition of CYP1A2. Indeed it is known that phenacetin incubated under non-saturated conditions (<5 μM) is mostly metabolised by CYP1A2 and partly by CYP1A1.

Furafylline preincubated for 30 minutes from 5 μM to 10 μM with human recombinant CYP1A2s brings about 100% inhibition of the purely CYP1A2 activity of the phenacetin, thereby proving Its maximum inhibition power under the selected conditions (FIG. 2).

When phenacetin Is incubated at a concentration very much greater than Its Km for CYP1A2 in the presence of Irreversibly inhibited microsomes and the control batch prepared under the conditions above, the percentage inhibition of the phenacetin deacetylase activity (CYP1A1-/CYP1A2-dependant) is still about 80%. This result proves that the inhibition of CYP1A2 by furafylline is not affected by an excess of substrate and that no competitive-type inhibition is detectable.

The specific substrates of the other major CYP450 (CYP2A6, 286, 2C8, 2C9, 2C19, 2D6, 2E1, and 3A4) were Incubated in the presence of the batch of human livers irreversibly inhibited by furafylline and of the control batch defined above in order to demonstrate the specificity of furafylline. It is observed that the activity of the other major CYP450s remains unchanged.

The entirety of these results shows that the microsomes isolated and Irreversibly and specifically inhibited with regard to CYP1 A2 according to the invention can be valuably used in measuring the contribution of CYP1 A2 to the metabolism of an active ingredient or drug candidate.

CYP3A4

Azamulin is one of the MBI inhibitors of the cytochrome CYP3A4.

The experimental conditions for maximum MBI inhibition by azamulin in the presence of CYP3A4 are as follows:
microsomal proteins at 2 mg/ml;
azamulin à 5 μM;
pre-incubation time of 15 minutes.

After incubation of midazolam at 0.5 μM, testosterone at 30 μM and nifedipine at 10 μM (concentrations less than or equal to the Km of the substrates), specific substrates of CYP3A4, in the presence of Irreversibly inhibited microsomes and of the control batch prepared under the conditions detailed above, the inhibition percentages of the midazolam-1′-hydroxylase, testosterone-B-hydroxylase and nifedipine-reductase (CYP3A4-/CYP3A5-dependant) activities are 81%, 96% and 83% respectively (FIG. 3). It Is to be noted that the remaining 19%, 4% and 17% metabolism is due to the CYP3A5 activity and not to a lack of inhibition of CYP3A4. Indeed it is known that the three specific substrates (and more especially nifedipine and midazolam) are metabolised mainly by CYP3A4 but also by CYP3A5. FIG. 4 especially shows the metabolism of nifedipine by recombinant microsomes (bactosomes) CYP3A4 and CYP3A5. Azamulin pre-incubated for 15 minutes at 5 μM with human recombinant CYP3A4s brings about 92% Inhibition of the purely CYP3A4 activity of midazolam, proving its maximum inhibition power under the conditions selected. When the specific substrates of CYP3A4 (using the example of midazolam) are Incubated at a concentration very much greater than their Km for CYP3A4 with microsomes previously prepared in accordance with the present invention, the percentage inhibition of the CYP3A4-dependant activities remains unchanged (FIG. 5). This result proves that the inhibition of CYP3A4 by azamulin is not affected by an excess of substrate and that no competitive-type inhibition is detectable.

The specific substrates of the other major CYP450s (CYP1A2, CYP2A6, 286, 208, 2C9, 2C19, 206, and 2E1) were incubated in the presence of the batch of human liver microsomes Irreversibly inhibited by azamulin and of the control batch under the conditions defined above in order to demonstrate the specificity of azamulin. FIG. 3 shows that the activity of the other main CYP450s remains unchanged between the batch of Irreversibly inhibited human microsomes and the control batch, demonstrating that azamulin is indeed specific for the CYP3A4 activity.

The entirety of these results shows that the microsomes isolated and irreversibly and specifically inhibited with regard to CYP3A4 according to the Invention can be valuably used in measuring the contribution of CYP3A4 to the metabolism of an active ingredient or drug candidate.

CYP2C8

Gemfibrozil glucuronide is one of the MBI inhibitors of the cytochrome CYP2C8. The experimental conditions for maximum MBI Inhibition by gemfibrozil glucuronide in the presence of CYP2C8 are as follows:
microsomal proteins at 2 mg/ml;
gemfibrozil glucuronide at 30 μM;
pre-incubation time of 30 minutes.

After incubation of amodiaquine at 0.5 μM or paclitaxel at 4 μM (concentrations less than or equal to the Km of the two substrates of CYP2C8), specific substrates of CYP2C8, in the presence of irreversibly Inhibited microsomes and of the control batch prepared according to the present invention, the percentage inhibition of the amodiaquine (CYP2C8) and paclitaxel-hydroxylase (CYP2C8-dependant) activities Is 88% and 100% (FIG. 6), respectively.

When amodiaquine is incubated at a concentration very much greater than its Km for CYP2C8 under the same conditions as those described above, the percentage inhibition of amodiaquine hydroxylase activity remains unchanged (FIG. 7). This result proves that the Inhibition of CYP2C8 by amodiaquine is not affected by an excess of substrate and that no competitive-type inhibition is detectable.

The specific substrates of the other major CYP450s were Incubated under the conditions defined above in order to demonstrate the specificity of gemfibrozil glucuronide. FIG. 6 shows that the activity of the other major CYP450s remains unchanged except for slight inhibition of CYP2C19 between the batch of Irreversibly inhibited human microsomes and the control batch, demonstrating that gemfibrozil glucuronide is Indeed specific for the CYP2C8 activity.

The entirety of these results shows that the microsomes isolated and irreversibly inhibited with regard to CYP2C8 according to the Invention can be valuably used in measuring the contribution of CYP2C8 to the metabolism of an active ingredient or drug candidate.

CYP2C9

Tienilic acid is one of the MBI inhibitors of the cytochrome CYP2C9.

The experimental conditions for maximum MBI inhibition by tienilic acid in the presence of CYP2C9 are as follows:
microsomal proteins at 2 mg/ml;
tienilic acid at 10 μM;
pre-incubation time of 20 minutes.

After incubation of diclofenac, a specific substrate of CYP2C9, at 4 M (concentration less than or equal to the Km of the substrate for CYP2C9) and 100 μM (concentration very much greater than its Km for CYP2C9) in the presence of irreversibly inhibited microsomes and of the control batch prepared according to the present invention, the inhibition of diclofenac hydroxylase (CYP2C9-dependant) activity Is almost total, or 92% and 88% inhibition respectively (FIG. 8). This result proves that not only is the CYP2C9 inhibition total but also that it is not affected by an excess of substrate and that no competitive-type inhibition is detectable.

The specific substrates of the other major CYP450s were Incubated under the conditions defined above in order to demonstrate the specificity of tienilic acid. FIG. 9 shows that the activity of the other major CYP450s remains unchanged between the batch of irreversibly Inhibited microsomes and the control batch, demonstrating that tienllic acid is indeed specific for the CYP2C9 activity.

The entirety of these results shows that the microsomes isolated and Irreversibly Inhibited with regard to CYP2C9 according to the Invention can be valuably used in measuring the contribution of CYP2C9 to the metabolism of an active ingredient or drug candidate.

CYP2D6

Paroxetine is one of the MBI inhibitors of the cytochrome CYP2D6.

The experimental conditions for maximum MBI inhibition by paroxetine in the presence of CYP2D6 are as follows:
microsomal proteins at 2 mg/ml;
paroxetine a 50 μM;
pre-incubation time of 30 minutes.

After incubation of dextromethorphan, a specific substrate of CYP2D6, at 5 μM (concentration less than or equal to the Km of the substrate) and 50 μM (concentration very much greater than its Km for CYP2D6) in the presence of irreversibly inhibited microsomes and of the control batch prepared according to the present Invention, the Inhibition of the dextromethorphan-O-demethylase (CYP2D6-dependant) activity is almost total, or 96% inhibition (FIG. 10). This result proves that the inhibition of CYP2D6 is total, that it Is not affected by an excess of substrate and that no competitive-type inhibition Is detectable.

The specific substrates of the other major CYP450s were incubated under the conditions defined above in order to demonstrate the specificity of paroxetine. FIG. 10 shows that, among all the other CYP450 activities, only the bupropion hydroxylase-dependant activity of CYP2B6 is inhibited, by 91%, In addition to CYP206, demonstrating that paroxetine is not completely specific for the CYP206 activity.

The entirety of these results shows that microsomes prepared according to the invention make it possible to inhibit CYP2D6 activity totally and almost specifically. They can therefore be used in measuring the contribution of CYP2D6/CYP2B6 to the metabolism of a new drug candidate.

CYP2B6

ThioTEPA Is one of the MBI Inhibitors of the cytochrome CYP2B6.

The experimental conditions for maximum MBI Inhibition by thioTEPA in the presence of CYP2B6 are as follows:
microsomal proteins at 2 mg/ml;
thioTEPA at 15 μM;
pre-incubation time of 30 minutes.

After incubation of bupropion, a specific substrate of CYP2B6, at 100 μM in the presence of irreversibly Inhibited microsomes and of the control batch prepared according to the present invention, inhibition of the bupropion hydroxylase (CYP2B6-dependant) activity Is almost total, or 92% Inhibition (FIG. 11). This result proves that the inhibition of CYP2B6 is total, that it is not affected by an excess of substrate and that no competitive-type inhibition is detectable.

The specific substrates of the other major CYP450s were incubated under the conditions defined above in order to demonstrate the specificity of thioTEPA. FIG. 11 shows that, among all the other CYP450 activities, only the coumarin hydroxylase-dependant activity of CYP2A6 is inhibited, by 64%, In addition to CYP2B86, demonstrating that thioTEPA is not completely specific for the CYP2B6 activity.

The entirety of these results shows that microsomes prepared according to the invention make it possible to inhibit CYP2B6 activity totally and almost specifically. They can therefore be used in measuring the contribution of CYP2B6/CYP2A6 to the metabolism of a new drug candidate.

### EXAMPLE 3: STABILITY OF THE INHIBITION OF CYTOCHROMES P450 IN MICROSOMES THAT HAVE BEEN ISOLATED, IRREVERSIBLY INHIBITED AND PRESERVED BY FREEZING

Isolated human liver microsomes irreversibly inhibited in terms of CYP1A2, having been concentrated and preserved at −80° C. in accordance with the Invention, are incubated for 15 minutes with phenacetin (4.5 μM), a specific substrate of CYP1A2, at 1 mg/mL The inhibition percentages were measured for microsomes obtained according to the invention and stored at −80° C. for 48 hours, one month, and one and a half months (FIG. 12). It Is observed that the steps of concentration and freezing/thawing do not affect the MBI Inhibition of CYP1A2 by furafylline.

The steps of freezing and thawing do not influence the stability of the Irreversible inhibition of the cytochromes P450.

### EXAMPLE 4: KIT OF IRREVERSIBLY INHIBITED ISOLATED MICROSOMES FOR ENZYMATIC PHENOTYPING OF THE METABOLIC PATHWAYS OF XENOBIOTICS

Nine active Ingredients (mirtazapine, loperamide, bupropion, ibuprofen, celocoxib, pioglitazone, bortezomib, repaglinide, sertraline) were tested in a kit of irreversibly Inhibited isolated microsomes according to the invention for enzymatic phenotyping of the metabolic pathways of said nine xenobiotics. The nine active ingredients were tests according to the method of phenotyping enzymatic reactions according to the invention comprising the following steps:
Incubation of microsomes Isolated and Irreversibly inhibited according to the invention with an active Ingredient to be evaluated;
measurement of the contribution of the irreversibly Inhibited cytochrome P450 Involved in the metabolism of the active Ingredient.

Each active ingredient was incubated at 0.1 μM at 37° C. in Tris/HCl buffer (0.1 mM, pH 7.4), with MgCl₂ 5 mM added, In the presence of, on the one hand, the Isolated liver microsomes irreversibly inhibited for the CYP450s 1A2, 286, 2C8, 2C9, 2D6 and 3A4 and, on the other hand, isolated non-Inhibited liver microsomes prepared in accordance with the Invention (homologous control). The reaction is initiated by addition of NADPH 1 mM. The incubation is monitored in kinetic form. At the incubation timepoints of 7 min, 17 min, 30 min and then 60 min, an incubation aliquot (100 μL) is sampled and the enzymatic reaction is stopped by adding to that aliquot a volume of solvent (100 μL of methanol) which is placed in ice for 10 minutes.

At each incubation timepoint, the active ingredient Is quantified by high-performance liquid chromatography (HPLC) coupled with mass spectrometry (MS).

The metabolic activity (A) of the active ingredient is measured via intrinsic metabolic clearance of the unchanged active Ingredient under the two conditions of inhibited (activity Ai) and non-inhibited (control with activity A).

The calculated percentage inhibition (percentage Inhibition=(A−Ai)/A) corresponds directly to the contribution of the irreversibly inhibited cytochrome P450 involved in the metabolism of the active ingredient.

Mirtazapine

Mirtazapine was incubated under the previously described conditions with a concentration of microsomal proteins of 2 mg/ml allowing optimum measurement of its intrinsic clearance. In the presence of control microsomes (non-inhibited and prepared according to the invention), an Intrinsic clearance of from 3.9 to 7.8 ml/min/g of proteins was measured. Compared to the control microsomes, inhibition of the intrinsic clearance of mirtazapine of 41%, 36% and 24% was found in the presence of isolated liver microsomes Irreversibly inhibited for the CYP450s 1A2, 2D6 and 3A4 respectively (FIGS. 13 and 14). No significant Inhibition of the Intrinsic clearance of mirtazapine was observed in the presence of Isolated liver microsomes Irreversibly Inhibited for the CYP450s 2B6, 208, 2C9. Significant inhibition is understood to be intrinsic clearance of less than 25%, a percentage representing the threshold of variability observed in clearance measurements on liver microsomes. Consequently, the oxidative metabolism of mirtazapine involves the CYP450s 1A2, 206 and 3A4 at levels of 41%, 36% and 24%, respectively.

After incubation of mirtazapine at from 2.5 to 1000 μM In the presence of recombinant microsomes overexpressing the major human CYP450s and after having measured the correction factor appropriate to each of those CYP450s, Störmer et al. (Metabolism of the antidepressant mirtazapine in vitro: contribution of cytochromes P-450 1A2, 206 and 3A4. Drug Metab Dispos. 2000; 28(10): 1168-1175) showed that the CYP450s 1A2, 216 and 3A4 exhibited 41%, 39% and 23% involvement, respectively, in the metabolism of mirtazapine. The results obtained with the kit of microsomes Isolated and Irreversibly Inhibited according to the Invention are corroborated by the results obtained by Störmer et al. (Table 1).

The kit according to the present invention allows the involvement of the CYP450s in the oxidative metabolism of mirtazapine to be deduced by simple comparison of the intrinsic clearances in the presence of isolated liver microsomes irreversibly inhibited for the CYP450 and of control microsomes.

In contrast, the use of recombinant microsomes overexpressing human CYP450s for the phenotyping of the enzymatic pathways of mirtazapine requires an Indirect measurement which requires each CYP450 activity to be characterised on the one hand in the presence of mirtazapine and on the other hand in the presence of specific substrates firstly recombinant microsomes overexpressing human CYP450s and secondly human liver microsomes in order to measure the correction factor.

TABLE 1
Percentage involvement for CYP450s in the oxidative metabolism
of mirtazapine obtained starting from the kit of isolated and
irreversibly inhibited microsomes according to the invention
and with human recombinant enzymes (Stömer et al.)

% involvement of CYP450s in the oxidative
metabolism of mirtazapine

| CYP450 | Kit of isolated | Expected |
|---|---|---|
| involved | microsomes | data* |
| CYP1A2 | 41 | 41 |
| CYP2D6 | 36 | 39 |
| CYP3A4 | 24 | 23 |

*from Stömer et al (2000) in a human recombinant enzyme mode

Loperamide

Loperamide was incubated under the previously described conditions with a concentration of microsomal proteins of 2 mg/ml allowing optimum measurement of its intrinsic clearance. In the presence of control microsomes (non-inhibited and prepared according to the invention), an intrinsic clearance of from 14.5 to 17.2 ml/min/g of proteins was measured. Compared to the control microsomes, inhibition of the intrinsic clearance of loperamide of 53% and 40% was found in the presence of isolated liver microsomes irreversibly inhibited for the CYP450s 3A4 and 2C8 respectively (FIGS. 15 and 16). No significant inhibition of the intrinsic clearance of loperamide was observed in the presence of isolated liver microsomes irreversibly Inhibited for the CYP450s 1A2, 2B6, 2C9, 206. Significant inhibition is understood to be intrinsic clearance of less than 25%, a percentage representing the threshold of variability observed in clearance measurements on liver microsomes. Consequently, the oxidative metabolism of loperamide involves the CYP450s 3A4 and 2C8 at levels of 53% and 40%, respectively.

A healthy volunteer study shows that the oral administration of gemfibrosil at 600 mg, an inhibitor of CYP2C8, increases, by a factor of 2.2, the exposure (AUC) to loperamide co-administered per os at 4 mg (Niemi et al. Itraconazole, gemfibrozil and their combination markedly raise the plasma concentrations of loperamide. Eur J Clin Pharmacol. 2006; 62: 463-472). This increase in exposure corresponds to an estimated involvement of CYP2C8 of 55% in the total loperamide clearance. In this same in vivo study, co-administration of loperamide 4 mg with 100 mg of Itraconazole, an inhibitor of CYP3A4, shows an increase in exposure by a factor of 3.8, corresponding to about 74% of the total loperamide clearance.

Furthermore, Tayrouz et al. (Ritonavir increases loperamide plasma concentrations without evidence for P-glycoprotein involvement. Clin Pharmacol Ther. 2001 November; 70(5):405-14) show that, in the healthy volunteer, the co-administration of loperamide 16 mg with 600 mg of ritonavir, an inhibitor of CYP3A4, causes an increase in exposure by a factor of 2.65, corresponding to 62% of the total loperamide clearance.

The results obtained with the kit of microsomes isolated and Irreversibly inhibited according to the invention (Table 2) are corroborated by the data described in a clinical situation by Niemi et al. and Tayrouz et al.

TABLE 2
Involvement percentages for CYP450s in the oxidative metabolism
of loperamide, which were obtained starting from the kit of isolated
and irreversibly inhibited microsomes according to the invention
and in healthy volunteers (Niemi et al.; Tayrouz et al.)

| | % involvement of CYP450s in the oxidative | | |
|---|---|---|---|
| | metabolism of loperamide | | |
| CYP450 | Kit of isolated | Data | Data |
| involved | microsomes | expected ¹ | expected² |
| CYP2C8 | 40 | 55 | |
| CYP3A4 | 53 | 74 | 62 |

¹ from Niemi et al. (2006) in an in vivo study in healthy volunteers
²from Tayrouz et al. (2001) in an in vivo study in healthy volunteers

Bupropion

Bupropion was incubated under the previously described, conditions with a concentration of microsomal proteins of 2 mg/ml allowing optimum measurement of its intrinsic clearance. In the presence of control is microsomes (non-inhibited and prepared according to the Invention), an intrinsic clearance of from 6.7 to 10.6 ml/min/g of proteins was measured. Compared to the control microsomes, inhibition of the intrinsic clearance of bupropion of 89% was found in the presence of Isolated liver microsomes irreversibly inhibited for the CYP450 286 (FIGS. 17 and 18). Inhibition of the intrinsic clearance of bupropion of 84% in the presence of isolated liver microsomes irreversibly inhibited for CYP2D6 was also observed. In the knowledge that paroxetine, an MBI inhibitor of CYP2D6, is not specific and also inhibits CYP2B6, it is deduced that the CYP2D6 Inhibition corresponds in reality to that of CYP2B6.

No significant Inhibition of the intrinsic clearance of bupropion was observed in the presence of isolated liver microsomes irreversibly inhibited for the CYP450s 1A2, 2C8, 2C9 and 3A4. Significant inhibition is understood to be intrinsic clearance of less than 25%, a percentage representing the threshold of variability observed in clearance measurements on liver microsomes. Consequently, the oxidative metabolism of bupropion involves the CYP450 2B6 at a level of 89%.

The FDA suggests bupropion as the most sensitive substrate for CYP2B6 in in vivo interaction studies in humans (FDA Website on Drug Development and Drug interactions, http://www.fda.gov/Drugs/GuidanceCompllanceRegulatorylnformaton/Guidances/default.htm and http://www.fda.gov/Drugs/DevelopmentApprovalProcess/evelopmentResources/DrugInteractioneLabelin 1277 glucm080499.htm).

The results obtained with the kit of microsomes isolated and Irreversibly Inhibited according to the invention are corroborated by the data described by the FDA.

TABLE 3
Involvement percentage for CYP450s in the oxidative metabolism
of bupropion, obtained starting from the kit of isolated and
irreversibly inhibited microsomes according to the invention

% involvement of CYP450s in the oxidative
metabolism of bupropion

| CYP450 | Kit of isolated | Expectd |
|---|---|---|
| involved | microsomes | data* |
| CYP2B6 | 89 | substantial |

*from the FDA (sensitive substrate CYP2B6)

Ibuprofen

Ibuprofen was Incubated under the previously described conditions with a concentration of microsomal proteins of 0.25 mg/ml allowing optimum measurement of its intrinsic clearance. In the presence of control microsomes (non-inhibited and prepared according to the invention), an intrinsic clearance of from 31 to 54 ml/min/g of proteins was measured. Compared to the control microsomes, inhibition of the intrinsic clearance of ibuprofen of 90% was found in the presence of isolated liver microsomes irreversibly inhibited for the CYP450 2C9 (FIGS. 19 and 20).

No significant inhibition of the intrinsic clearance of ibuprofen was observed in the presence of isolated liver microsomes irreversibly Inhibited for the CYP450s 1A2, 2B6, 2D6, 2C8 and 3A4. Significant Inhibition is understood to be intrinsic clearance of less than 25%, a percentage representing the threshold of variability observed in clearance measurements on liver microsomes. Consequently, the oxidative metabolism of loperamide involves the CYP450 2C7 at a level of 90%.

After incubation of Ibuprofen at 3 μM In the presence of recombinant microsomes overexpressing the major human CYP450s and after having measured the correction factor appropriate for each of those CYP450s, McGinnity et al. (Automated definition of the enzymology of drug oxidation by the major human drug metabolizing cytochrome P450s. Drug Metab Dispos. 2000 November; 28(11):1327-34) showed that the CYP450 2C9 was involved at a level of 90% in the metabolism of Ibuprofen. The results obtained with the kit of isolated and Irreversibly Inhibited microsomes according to the Invention are corroborated by the data described by McGinnity et al. (Table 4). It will be recalled that the kit according to the present invention establishes the involvement of the CYP450 in the oxidative metabolism of Ibuprofen by simple comparison between the Intrinsic clearances of microsomes according to the invention and control microsomes. In contrast, the use of recombinant microsomes necessitates indirect measurements which require multiplication of procedures.

Furthermore, a study in healthy volunteers shows that the oral administration of fluconazole at 400 mg increases the exposure (AUC) to Ibuprofen co-administered per os at 400 mg by 883% (Hynninen et a. Effects of the Antifungals Voriconazole and Fluconazole on the Pharmacokinetics of S-(+)- and R-(−)-Ibuprofen. Antimicrob Agents Chemother. June 2006; 50(6): 1967-1972). Lazar et al (Drug interactions with fluconazole. Rev Infect Dis. 1990 March-April; 12 Suppl 3:S327-33) have shown that fluconazole, an inhibitor of CYP2C9, brings about a 109% increase in exposure to tolbutamide, a substrate recognised as being sensitive to CYP2C9 (fm=80%, Brown et al. Prediction of in vivo drug-drug interactions from in vitro data: Impact of Incorporating parallel pathways of drug elimination and inhibitor absorption rate constant. Br J Clin Pharmacol. 2005 November; 60(6):508-18). As the increase in the exposure to Ibuprofen and tolbutamide is very similar after co-administration of the same inhibitor in humans, it is possible to conclude that the contribution of CYP2C9 to the metabolism of these two molecules is very similar. The results obtained with the kit of the present invention are confirmed (Table 4) and demonstrate the excellent representativity of this in vitro model compared to the clinical situation.

TABLE 4
Percentage involvement of CYP450s in the oxidative metabolism
of ibuprofen, starting from the kit of isolated and irreversibly
inhibited microsomes, in the presence of recombinant human enzymes
(McGinnity et al.) and in an in vivo situation in healthy subjects
(Hynninen et al., Lazar et al. and Brown et al.).

| | | % involvement of CYP450s in the oxidative | | | |
|---|---|---|---|---|---|
| | Kit of | metabolism of ibuprofen | | | |
| CYP450 | isolated | Expected | Expected | Expected | Expected |
| involved | microsomes | data ¹ | data ² | data³ | data ⁴ |
| CYP2C9 | 90 | 91 | 45 | 52 | (80) |

¹ from McGinnity et al. (2000) in a model of recombinant human enzymes
² from Hynninen at al. (2006)
³from Lazar J D et al. (1990)
⁴ from H. S. Brown et al. (2005)

Celocoxib

Celocoxib was Incubated under the previously described conditions with a concentration of microsomal proteins of 2 mg/ml allowing optimum measurement of its Intrinsic clearance. In the presence of control microsomes (non-inhibited and prepared according to the invention), an intrinsic clearance of from 13.4 to 18.9 ml/min/g of proteins was measured. Compared to the control microsomes, inhibition of the intrinsic clearance of celocoxib of 81% was found in the presence of isolated liver microsomes irreversibly inhibited for the CYP450 2C09 (FIGS. 21 and 22). No significant inhibition of the Intrinsic clearance of celocoxib was observed in the presence of isolated liver microsomes irreversibly inhibited for the CYP450s 1A2, 2B6, 2D6, 208 and 3A4. Significant Inhibition is understood to be intrinsic clearance of less than 25%, a percentage representing the threshold of variability observed in clearance measurements on liver microsomes. Consequently, the oxidative metabolism of celocoxib involves the CYP450 209 at a level of 81%.

A healthy volunteer study shows that the repeated oral administration of fluconazole at 200 mg increases, by 134%, the exposure (AUC) to celocoxib co-administered per os at 200 mg (NDA020996 1998-12-31 Pharmacia).

As the Increase in the exposure to celocoxib and to the above-mentioned tolbutamide (Lazar at al.) is very similar after co-administration of fluconazole in humans, it is possible to conclude that the contribution of CYP2C9 to the metabolism of these two active Ingredients is very similar. Brown et al. have shown for tolbutamide an involvement of CYP2C9 of 80%. The results obtained with the kit of isolated microsomes irreversibly inhibited according to the invention demonstrate the good representativity of this in vitro model compared to the clinical situation (Table 5).

TABLE 5
Percentage involvement of CYP450s in the oxidative metabolism of
celocoxib, starting from the kit of isolated microsomes irreversibly
inhibited according to the invention and in an in vivo situation
in healthy subjects (NDA020998, Lazar et al. and Brown et al.)

| | % involvement of CYP450s in the oxidative | | | |
|---|---|---|---|---|
| | metabolism of celocoxib | | | |
| CYP450 | Kit of isolated | Expected | Expected | Expected |
| involved | microsomes | data ¹ | data ² | data ³ |
| CVP2C9 | 81 | 57 | 52 | (80) |

¹from NDA020998 1998 Dec. 31 (Pharmacia)
²from Lazar J D et al. (1990)
³from H. S. Brown et al. (2005)

Pioglitazone

Pioglitazone was incubated under the previously described conditions with a concentration of microsomal proteins of 0.2 mg/ml allowing optimum to measurement of Its Intrinsic clearance. In the presence of control microsomes (non-inhibited and prepared according to the Invention), an intrinsic clearance of from 43 to 70 ml/min/g of proteins was measured. Compared to the control microsomes, inhibition of the Intrinsic clearance of pioglitazone of 69% was found in the presence of isolated liver microsomes irreversibly inhibited for the CYP450 2C8 (FIGS. 23 and 24).

No significant inhibition of the intrinsic clearance of pioglitazone was observed in the presence of isolated liver microsomes irreversibly inhibited for the CYP450s 1A2, 2B6, 2D6, 2C9 and 3A4. Significant inhibition is understood to be intrinsic clearance of less than 25%, a percentage representing the threshold of variability observed in clearance measurements on liver microsomes. Consequently, the oxidative metabolism of pioglitazone involves the CYP450 2C8 at a level of 69%.

A healthy volunteer study shows that the repeated oral administration of gemfibrosil, an inhibitor of CYP2C8, at 600 mg increases, by 239%, the exposure (AUC) to pioglitazone co-administered per os at 3 mg (Deng at al. Effect of gemfibrozil on the pharmacokinetics of pioglitazone. Eur J Clin Pharmacol, 2005, 61, 831-6). This Increase in exposure corresponds to involvement of CYP2C8 estimated to be 71% of the total clearance of pioglitazone. The results obtained with the kit according to the invention therefore corroborate the data described in a clinical situation by Deng at al. (Table 6).

TABLE 6
Percentage involvement of CYP450s in the oxidative metabolism
of pioglitazone, obtained starting from the kit of isolated
microsomes irreversibly inhibited according to the invention
and in an in vivo situation (Deng et al.)

% involvement of CYP450s in the oxidative
metabolism of pioglitazone

| CYP450 | Kit of isolated | Expected |
|---|---|---|
| involved | microsomes | data¹ |
| CYP2C8 | 69 | 71 |

¹from Deng L J et al., 2005, in vivo study

Bortezomib

Bortezomib was Incubated under the previously described conditions with a concentration of microsomal proteins of 1.5 mg/ml allowing optimum measurement of its intrinsic clearance. In the presence of control microsomes (non-inhibited and prepared according to the invention), an intrinsic clearance of from 6.9 to 11 ml/min/g of proteins was measured. Compared to the control microsomes, inhibition of the Intrinsic clearance of bortezomib of 73% was found in the presence of isolated liver microsomes Irreversibly inhibited for the CYP450 3A4 (FIGS. 25 and 26).

No significant inhibition of the intrinsic clearance of bortezomib was observed in the presence of isolated liver microsomes irreversibly inhibited for the CYP450s 1A2, 2B6, 2D6, 2C8 and 209. Significant inhibition is understood to be intrinsic clearance of less than 25%, a percentage representing the threshold of variability observed in clearance measurements on liver microsomes. Consequently, the oxidative metabolism of bortezomib Involves the CYP450 3A4 at a level of 73%.

Uttamsingh et al. (Relative contributions of the five major human cytochromes p450, 1A2, 209, 2019, 2D6, and 3A4, to the hepatic metabolism of the proteasome Inhibitor bortezomib. Drug Metab Dipos 2005, 33 (11):1723-1728) have shown that an anti-CYP 3A4 monoclonal antibody inhibits 79% of the metabolism of bortezomib (2 μM) by human liver microsomes. The results obtained with the kit described in the present invention therefore corroborate the data described by Uttamsingh et al. (Table 7).

TABLE 7
Percentage involvement of CYP450s in the oxidative metabolism
of borezomib, obtained starting from the kit of isolated
microsomes irreversibly inhibited according to the invention
and starting from human hepatic microsomes inhibited by
specific monoclonal antibodies (Uttamsingh et al.)

% involvement of CYP450s in the oxidative
metabolism of bortezomib

| CYP450 | Kit of isolated | Expected |
|---|---|---|
| involved | microsomes | data¹ |
| CYP3A4 | 73 | 79 |

¹from Uttamsingh et al. (2005) in a model of human hepatic microsomes (use of monoclonal antibodies)

Repaglinide

Repaglinide was incubated under the previously described conditions with a concentration of microsomal proteins of 2 mg/ml allowing optimum measurement of Its Intrinsic clearance. In the presence of control microsomes (non-inhibited and prepared according to the invention), an Intrinsic clearance of from 38.4 to 48.9 ml/min/g of proteins was measured. Compared to the control microsomes, inhibition of the intrinsic clearance of repaglinide of 80% was found in the presence of isolated liver microsomes irreversibly inhibited for the CYP450 2C8 (FIGS. 27 and 28). No significant inhibition of the intrinsic clearance of repaglinide was observed in the presence of Isolated liver microsomes irreversibly Inhibited for the CYP460s 1A2, 2B6, 2D6, 2C9 and 3A4. Significant inhibition Is understood to be intrinsic clearance of less than 25%, a percentage representing the threshold of variability observed in clearance measurements on liver microsomes. Consequently, the oxidative metabolism of repaglinide Involves the CYP450 2C8 at a level of 80%.

A healthy volunteer study shows that the oral administration of gemfibrosil (up to 900 mg), an inhibitor of CYP2C8, increases, by 8.3 times, the exposure (AUC) to repaglinide co-administered per os at 0.25 mg (Honkalammi J. et al. Dose-Dependent Interaction between gemfibrozil and repaglinide in humans: strong Inhibition of CYP2C8 with subtherapeutic gemfibrozil doses. Drug Metab Dispos, 2011, 39, 1977-1966). This increase in exposure corresponds to involvement of CYP2C8 estimated to be 88% of the total clearance of repaglinide. The results obtained with the kit of isolated microsomes Irreversibly inhibited according to the Invention are corroborated by the data described in a clinical situation by Honkalammi J. et al. (Table 8).

TABLE 8
Percentage involvement of CYP450s in the oxidative metabolism of
repaglinide, obtained starting from the kit of isolated microsomes
irreversibly inhibited according to the invention and in an in
vivo situation in healthy subjects P (Honkalammi J. et al.)

% involvement of CYP450s in the oxidative
metabolism of repaglinide

| CYP450 | Kit of isolated | Expected |
|---|---|---|
| involved | microsomes | data ¹ |
| CYP2C8 | 80 | 88 |

¹ from Honkálammi J. et al., 2011.

Sertraline

Sertraline was incubated under the previously described conditions with a concentration of microsomal proteins of 0.2 mg/ml allowing optimum measurement of its intrinsic clearance. In the presence of control microeomes (non-inhibited and prepared according to the Invention), an Intrinsic clearance of from 52.5 to 70.5 m/min/g of proteins was measured. Compared to the control microsomes, inhibition of the Intrinsic clearance of sertraline of 58% was found in the presence of isolated liver microsomes Irreversibly inhibited for the CYP450 2B6 (FIGS. 29 and 30). Inhibition of the intrinsic clearance of sertraline of 64% in the presence of isolated liver microsomes Irreversibly Inhibited for CYP2D6 was also observed. In the knowledge that paroxetine, an MBI inhibitor of CYP2D6, is not specific and also inhibits CYP2B6, it is deduced that the CYP2D6 Inhibition corresponds in reality to that of CYP2B6.

No significant inhibition of the intrinsic clearance of sertraline was observed in the presence of isolated liver microsomes irreversibly Inhibited for the CYP450s 1A2, 2C8, 2C9 and 3A4. Significant inhibition is understood to be Intrinsic clearance of less than 25%, a percentage representing the threshold of variability observed in clearance measurements on liver microsomes. Consequently, the oxidative metabolism of sertraline involves the CYP450 286 at a level of 58%.

After incubation of sertraline in the presence of human liver microsomes and specific inhibitors of CYP450s, Obach S at al. (Sertraline is metabolized by multiple cytochrome P450 enzymes, monoamine oxidases, and glucuronyl transferases in human: an in vitro study. Drug Metab Dispos. 2005 February; 33(2):262-70) have shown that, among the major CYP450s, CYP2B6 contributes most to the metabolism of sertraline with an involvement of from 15 to 65% (60% in a pool of human liver). The results obtained with the kit described according to the present invention are corroborated by the results described by Obach S et al. (Table 9).

TABLE 9
Percentage involvement of CYP450s in the oxidative metabolism
of sertraline, obtained starting from the kit of isolated,
irreversibly inhibited microsomes and in human liver microsomes
in the presence, or not, of CYP450s (Obach S et al.)

% involvement of CYP450s in the oxidative
metabolism of sertraline

| CYP450 | Kit of isolated | Expected |
|---|---|---|
| involved | microsomes | data ¹ |
| CYP2B6 | 58 | 15 to 65-60 |

¹ from Obach S et al. (2004) in human liver microsomes +/− specific inhibitor of CYP450

The results obtained show that the contribution of enzymes involved in the metabolism of the selected active ingredients, measured in vitro using the phenotyping kit, are very similar or even Identical to those estimated or measured on the basis of in vivo data and/or data obtained from other in vitro models. Validation of the kit of Irreversibly inhibited, isolated end cryopreserved microsomes in the context of enzymatic phenotyping of the metabolic pathways of a xenobiotic compared to the clinical data demonstrates the representativity of this in vitro model compared to the in vivo situation in humans.

Furthermore, obtaining a direct measurement of the enzymatic contribution to the metabolism of an active ingredient not only makes possible a benefit in terms of time and facility of interpretation but also avoids errors which are inherent in the multiplication of manipulations in the carrying out of other in vitro models.

1. A method of preparing isolated microsomes comprising an irreversibly inhibited cytochrome P450 (CYP450), comprising the following steps:
a) irreversibly inhibiting a cytochrome P450;
b) concentrating the microsomal proteins; and
c) cryopreserving or freezing at −80° C.

2. The method of claim 1, further comprising one or more washing steps.

3. The method of claim 2, wherein the washing step or steps is/are performed before and/or after the step of concentrating the microsomal proteins.

4. The method of claim 1, wherein the microsomes are concentrated by filtration/centrifugation or ultracentrifugations.

5. The method of claim 1, wherein the microsomes are concentrated to a concentration of 10 mg/ml to 30 mg/ml.

6. The method of claim 1, comprising a final step of preserving the microsomal proteins.

7. The method of claim 6, wherein the final step of preserving is carried out by freezing.

8. The method of claim 1, wherein the microsomes are human liver microsomes.

9. The method of claim 1, wherein the irreversibly inhibited cytochrome P450 is selected from the group consisting of cytochrome CYP1, cytochrome CYP2 and cytochrome CYP3.

10. The method of claim 9, wherein cytochrome P450 is selected from the group consisting of cytochrome CYP1A2, cytochrome CYP2A6, cytochrome CYP2B6, cytochrome CYP2C8, cytochrome CYP2C9, cytochrome CYP2C19, cytochrome CYP2D6, cytochrome CYP2E1, and cytochrome CYP3A4.

11. An isolated and cryopreserved or frozen microsome, wherein a cytochrome P450 selected from the group consisting of cytochrome CYP1A2, cytochrome CYP2A6, cytochrome CYP2B6, cytochrome CYP2C8, cytochrome CYP2C9, cytochrome CYP2C19, cytochrome CYP2D6, cytochrome CYP2E1, and cytochrome CYP3A4 of the microsome is irreversibly inhibited.

12. An isolated and cryopreserved or frozen microsome obtained according to the method of claim 1, wherein a cytochrome P450 selected from the group consisting of cytochrome CYP1A2, cytochrome CYP2A6, cytochrome CYP2B6, cytochrome CYP2C8, cytochrome CYP2C9, cytochrome CYP2C19, cytochrome CYP2D6, cytochrome CYP2E1, and cytochrome CYP3A4 of the microsome is irreversibly inhibited.

13. A method of phenotyping enzymatic reactions involved in the metabolism of an active ingredient, comprising the following steps:
(a) incubating the isolated microsomes of claim 11 with an active ingredient; and
(b) measuring the contribution of the irreversibly inhibited cytochrome P450 involved in the metabolism of the active ingredient.

14. A phenotyping kit, comprising:
(a) the isolated microsomes of claim 11; and
(b) control microsomes.
